(19) United States
(12) Patent Application Publication
(10) Pub. No.: US 2025/0129057 A1
(43) Pub. Date: Apr. 24, 2025

(54) Solid Forms of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-Dihydroxy-2-(hydroxymethyl)propan-2-amine Salt

(71) Applicant: Pfizer Inc., New York, NY (US)

(72) Inventors: Scott W. Bagley, Voluntown, CT (US); Wesley Dewitt Clark, Gales Ferry, CT (US); David Andrew Griffith, Sudbury, MA (US); Wenhua Jiao, Salem, CT (US); Brian Matthew Samas, Branford, CT (US); Lisa Jane Taylor, Ramsgate, Kent (GB)

(73) Assignee: Pfizer Inc., New York, NY (US)

(21) Appl. No.: 18/685,644

(22) PCT Filed: Aug. 25, 2022
(86) PCT No.: PCT/IB2022/057973

§ 371 Date: Feb. 22, 2024

Related U.S. Application Data
(60) Provisional application No. 63/239,056, filed on Aug. 31, 2021.

(51) Int. Cl. C07D 405/14; A61K 31/4545

(52) U.S. Cl. CPC C07D 405/14; A61K 31/4545

(57) ABSTRACT

The invention provides solid forms of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt for example, Form 1 or Form 2; as well as pharmaceutical compositions, and the uses thereof in treating diseases, conditions or disorders modulated by GLP-1R in a mammal, such as a human.

This application is a national phase filing under 35 U.S.C. § 371 of international patent application number PCT/IB2022/057973 filed Aug. 25, 2022, which in turn claims the benefit of priority to U.S. Provisional Patent Application Ser. No. 63/239,056 filed Aug. 31, 2021, the disclosure of each of these applications is hereby incorporated by reference in its entirety.

### FIELD OF INVENTION

The invention provides solid forms (e.g. crystalline and/or amorphous forms) of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt; processes for preparing thereof; pharmaceutical compositions, dosage forms, and uses thereof in treating diseases, conditions or disorders modulated by GLP-1R in a mammal such as a human.

### BACKGROUND OF THE INVENTION

Diabetes is a major public health concern because of its increasing prevalence and associated health risks. The disease is characterized by high levels of blood glucose resulting from defects in insulin production, insulin action, or both. Two major forms of diabetes are recognized, Type 1 and Type 2. Type 1 diabetes (T1D) develops when the body's immune system destroys pancreatic beta cells, the only cells in the body that make the hormone insulin that regulates blood glucose. To survive, people with Type 1 diabetes must have insulin administered by injection or a pump. Type 2 diabetes mellitus (referred to generally as T2DM) usually begins with either insulin resistance or when there is insufficient production of insulin to maintain an acceptable glucose level.

Currently, various pharmacological approaches are available for treating hyperglycemia and subsequently, T2DM (Hampp, C. et al. Use of Antidiabetic Drugs in the U.S., 2003-2012, Diabetes Care 2014, 37, 1367-1374). These may be grouped into six major classes, each acting through a different primary mechanism: (A) Insulin secretogogues, including sulphonyl-ureas (e.g., glipizide, glimepiride, glyburide), meglitinides (e.g., nateglidine, repaglinide), dipeptidyl peptidase IV (DPP-IV) inhibitors (e.g., sitagliptin, vildagliptin, alogliptin, dutogliptin, linagliptin, saxogliptin), and glucagon-like peptide-1 receptor (GLP-1R) agonists (e.g., liraglutide, albiglutide, exenatide, lixisenatide, dulaglutide, semaglutide), which enhance secretion of insulin by acting on the pancreatic beta-cells. Sulphonyl-ureas and meglitinides have limited efficacy and tolerability, cause weight gain and often induce hypoglycemia. DPP-IV inhibitors have limited efficacy. Marketed GLP-1R agonists are peptides administered by subcutaneous injection. Liraglutide is additionally approved for the treatment of obesity. (B) Biguanides (e.g., metformin) are thought to act primarily by decreasing hepatic glucose production. Biguanides often cause gastrointestinal disturbances and lactic acidosis, further limiting their use. (C) Inhibitors of alpha-glucosidase (e.g., acarbose) decrease intestinal glucose absorption. These agents often cause gastrointestinal disturbances. (D) Thiazolidinediones (e.g., pioglitazone, rosiglitazone) act on a specific receptor (peroxisome proliferator-activated receptor-gamma) in the liver, muscle, and fat tissues. They regulate lipid metabolism subsequently enhancing the response of these tissues to the actions of insulin. Frequent use of these drugs may lead to weight gain and may induce edema and anemia. (E) Insulin is used in more severe cases, either alone or in combination with the above agents, and frequent use may also lead to weight gain and carries a risk of hypoglycemia. (F) sodium-glucose linked transporter cotransporter 2 (SGLT2) inhibitors (e.g., dapagliflozin, empagliflozin, canagliflozin, ertugliflozin) inhibit reabsorption of glucose in the kidneys and thereby lower glucose levels in the blood. This emerging class of drugs may be associated with ketoacidosis and urinary tract infections.

However, with the exception of GLP-1R agonists and SGLT2 inhibitors, the drugs have limited efficacy and do not address the most important problems, the declining B-cell function and the associated obesity.

Obesity is a chronic disease that is highly prevalent in modern society and is associated with numerous medical problems including hypertension, hypercholesterolemia, and coronary heart disease. It is further highly correlated with T2DM and insulin resistance, the latter of which is generally accompanied by hyperinsulinemia or hyperglycemia, or both. In addition, T2DM is associated with a two to fourfold increased risk of coronary artery disease. Presently, the only treatment that eliminates obesity with high efficacy is bariatric surgery, but this treatment is costly and risky. Pharmacological intervention is generally less efficacious and associated with side effects. There is therefore an obvious need for more efficacious pharmacological intervention with fewer side effects and convenient administration.

Although T2DM is most commonly associated with hyperglycemia and insulin resistance, other diseases associated with T2DM include hepatic insulin resistance, impaired glucose tolerance, diabetic neuropathy, diabetic nephropathy, diabetic retinopathy, obesity, dyslipidemia, hypertension, hyperinsulinemia, and nonalcoholic fatty liver disease (NAFLD).

NAFLD is the hepatic manifestation of metabolic syndrome, and is a spectrum of hepatic conditions encompassing steatosis, non-alcoholic steatohepatitis (NASH), fibrosis, cirrhosis and ultimately hepatocellular carcinoma. NAFLD and NASH are considered the primary fatty liver diseases as they account for the greatest proportion of individuals with elevated hepatic lipids. The severity of NAFLD/NASH is based on the presence of lipid, inflammatory cell infiltrate, hepatocyte ballooning, and the degree of fibrosis. Although not all individuals with steatosis progress to NASH, a substantial portion does.

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. In non-clinical experiments GLP-1 promotes continued beta-cell competence by stimulating transcription of genes important for glucose-dependent insulin secretion and by promoting beta-cell neogenesis (Meier, et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with T2DM, the normal post-prandial rise in GLP-1 is absent or reduced (Vilsboll T, et al. Diabetes. 2001. 50; 609-613).

Holst (Physiol. Rev. 2007, 87, 1409) and Meier (Nat. Rev. Endocrinol. 2012, 8, 728) describe that GLP-1 receptor agonists, such as GLP-1, liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

There remains a need for an easily-administered prevention and/or treatment for cardiometabolic and associated diseases.

2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, or a pharmaceutically acceptable salt thereof [e.g. its 1,3-dihydroxy-2-(hydroxymethyl) propan-2-amine salt, also known as its 2-amino-2-(hydroxymethyl) propane-1,3-diol salt, or its tris(hydroxyethyl)methylamine salt, or its tris salt] is a GLP-1R agonist described in U.S. Pat. No. 10,208,019 (see Example 4A-01 of the patent), the disclosure of which is hereby incorporated by reference herein in its entirety for all purposes.

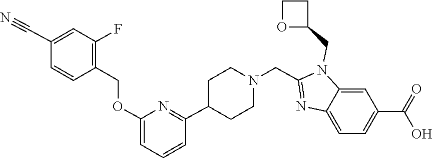 [SMILES: N#Cc1ccc(COc2cccc(C3CCN(Cc4nc5ccc(C(=O)O)cc5n4C[C@@H]4CCO4)CC3)n2)c(F)c1]

2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (“Compound 1”).

Tris salt of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid means a salt of Compound 1 made by using 1,3-dihydroxy-2-(hydroxymethyl) propan-2-amine. The tris is associated with the carboxylic acid moiety of Compound 1. Unless otherwise stated, when referencing the tris salt of Compound 1, the counterion and Compound 1 are in a stoichiometric ratio of about 1:1 (i.e. from 0.9:1.0 to 1.0:0.9, for example, from 0.95:1.00 to 1.00:0.95, or from 0.99:1.00 to 1.00:1.01). Another chemical name for tris salt of Compound 1 is 1,3-dihydroxy-2-(hydroxymethyl) propan-2-aminium 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylate, which can also be represented, for example, by one of the following structures.

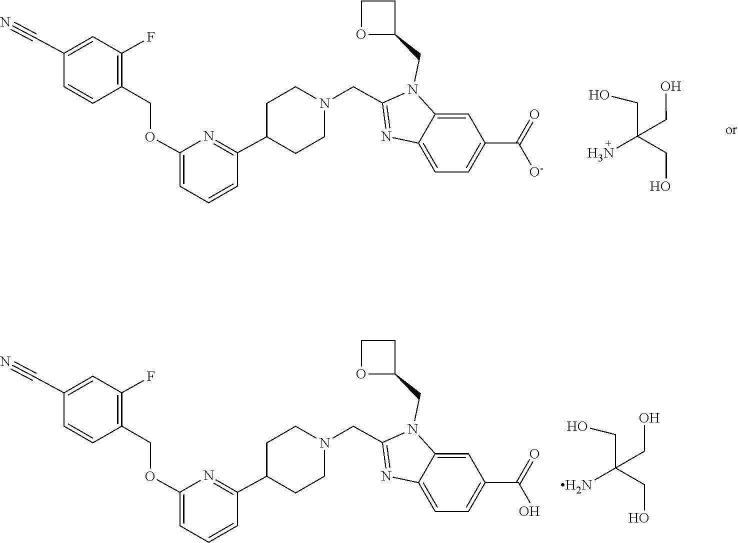

### Tris Salt of Compound 1

It is well known that a solid form, for example a crystalline form of a particular drug (including, e.g., anhydrate, hydrate, solvate, etc.) is often an important determinant of the drug's ease of preparation, stability, solubility, storage stability, ease of formulation, ease of handling, and in vivo pharmacology and/or efficacy. Different crystalline forms occur where the same composition of matter crystallizes in a different lattice arrangement resulting in different thermodynamic properties and stabilities specific to the particular polymorph form. In cases where two or more solid forms (e.g. two or more crystalline forms, or an amorphous form and one or more crystalline forms) can be produced, it is desirable to have a method to make each of the solid forms in pure form. In deciding which solid form is preferable, the numerous properties of the solid forms must be compared and the preferred solid (e.g. crystalline) form chosen based on the many physical property variables. It is entirely possible that one crystalline form can be preferable in some circumstances where certain aspects such as ease of preparation, stability, etc. are deemed to be critical. In other situations, a different crystalline form maybe preferred for greater solubility and/or superior pharmacokinetics. Moreover, because of the potential advantages associated with one pure crystalline form, it is desirable to prevent or minimize polymorphic conversion (i.e., conversion of one crystal form to another; or conversion between one crystal form and amorphous form) when two or more solid forms of one substance can exist. Such polymorphic conversion can occur during both the preparation of formulations containing a solid form (e.g. a crystalline form), and during storage of a pharmaceutical dosage form containing a solid form (e.g. a crystalline form). Because improved drug formulations showing, for example, better bioavailability or better stability are consistently sought, there is an ongoing need for new or purer solid (e.g. crystalline) forms of existing drug molecules. The novel solid forms (e.g. crystalline and/or amorphous forms) of tris salt of Compound 1 described herein are directed toward this and other important ends.

### SUMMARY OF THE INVENTION

In one embodiment (Embodiment A1), the present invention provides a crystalline form of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (e.g. Form 1). The crystalline form of the invention may be characterized according to the powder X-ray diffraction (PXRD) data, solid state Nuclear Magnetic Resonance (ssNMR) data (e.g. ¹³C ssNMR data), and/or FT-Raman spectroscopy data provided herein.

In one embodiment (Embodiment B1), the present invention provides an amorphous form of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (e.g. Form 2). The amorphous form of the invention may be characterized according to the powder X-ray diffraction (PXRD) data, solid state Nuclear Magnetic Resonance (ssNMR) data (e.g. ¹³C ssNMR data, ¹⁵N ssNMR data, and ¹⁹F ssNMR data), and/or FT-Raman spectroscopy data provided herein.

The present invention further provides a pharmaceutical composition containing a crystalline form of the invention (e.g. Form 1).

The present invention further provides a pharmaceutical composition containing an amorphous form of the invention (e.g. Form 2).

The present invention further provides a method for preparing Form 1 of tris salt of Compound 1 comprising precipitating (crystalizing) Form 1 from a solution, wherein the solution comprises tris salt of Compound 1 and a solvent, and wherein the solvent comprises 1-propanol.

The present invention further provides a method for preparing Form 1 of tris salt of Compound 1 comprising precipitating (crystalizing) the Form 1 from a solution, wherein the solution comprises tris salt of Compound 1 and a mixed solvent, and wherein the mixed solvent comprises water and DMSO (dimethyl sulfoxide).

The present invention further provides a method for preparing an amorphous form of the invention, for example, a method for preparing Form 2 of tris salt of Compound 1 comprising ball milling Form 1 of tris salt of Compound 1 to provide Form 2 of tris salt of Compound 1.

The present invention further provides a method for treating a disease or disorder comprising administering to a mammal in need of such treatment a therapeutically effective amount of a crystalline form of the invention (e.g. Form 1) or an amorphous form of the invention (e.g. Form 2), wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, a cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

The present invention further provides a crystalline form of the invention (e.g. Form 1) or an amorphous form of the invention (e.g. Form 2) for use in treating a disease or disorder modulated by GLP-1R.

The present invention further provides use of a crystalline form of the invention (e.g. Form 1) or an amorphous form of the invention (e.g. Form 2) for use as a medicament.

The present invention further provides use of a crystalline form of the invention (e.g. Form 1) or an amorphous form of the invention (e.g. Form 2) in treating a disease or disorder modulated by GLP-1R.

The present invention further provides a pharmaceutical combination comprising a therapeutically effective amount of a crystalline form of the invention (e.g. Form 1) and an additional agent.

The present invention further provides a pharmaceutical combination comprising a therapeutically effective amount of an amorphous form of the invention (e.g. Form 2) and (2) an additional agent.

### BRIEF DESCRIPTION OF FIGURES

FIG. 1 shows an observed powder X-ray diffraction pattern (PXRD) for Form 1 of tris salt of Compound 1 carried out on a Bruker AXS D8 Endeavor diffractometer equipped with a Cu Kα radiation source (wavelength of 1.5406 Å).

FIG. 2 shows an observed ¹³C ssNMR pattern of Form 1 of tris salt of Compound 1 conducted on a 4 mm magic angle spinning (MAS) probe at MAS rates of 10 KHz positioned into a Bruker Avance III HD 400 MHZ (1H frequency) NMR spectrometer.

FIG. 3 shows an observed ¹⁵N ssNMR pattern of Form 1 of tris salt of Compound 1 conducted on a Bruker AVANCE NEO 400 MHZ NMR spectrometer equipped with a 4 mM MAS probe with a spin rate of 20 KHz.

FIG. 4 shows an observed ¹⁹F ssNMR pattern of Form 1 of tris salt of Compound 1 conducted on a 3.2 mm MAS probe with a spin rate of 20 KHz positioned into a Bruker Avance III HD 400 MHz (1H frequency) NMR spectrometer.

FIG. 5 shows a representative, observed FT-Raman spectrum of tris salt of Compound 1, using a RAM II FT-Raman module attached to a Vertex 70 spectrometer (Bruker Optik GmbH).

FIG. 6 shows an observed powder X-ray diffraction pattern (PXRD) for Form 2 of tris salt of Compound 1 carried out on a Bruker AXS D8 Endeavor diffractometer equipped with a Cu Kα radiation source (wavelength of 1.5406 Å).

FIG. 7 shows an observed ¹³C ssNMR pattern of Form 2 of tris salt of Compound 1 conducted on a 4 mm magic angle spinning (MAS) probe at MAS rates of 10 kHz positioned into a Bruker Avance III HD 400 MHZ (1H frequency) NMR spectrometer.

FIG. 8 shows an observed ¹⁵N ssNMR pattern of Form 2 of tris salt of Compound 1 conducted on 4 mm magic angle spinning (MAS) probe at MAS rates of 8 kHz positioned into a Bruker Avance III HD 400 MHZ (1H frequency) NMR spectrometer.

FIG. 9 shows an observed ¹⁹F ssNMR pattern of Form 2 of tris salt of Compound 1 conducted on a 3.2 mm MAS probe with a spin rate of 20 KHz positioned into a Bruker Avance III HD 400 MHZ (1H frequency) NMR spectrometer.

FIG. 10 shows a representative, observed FT-Raman spectrum of Form 2 of tris salt of Compound 1, using a RAM II FT-Raman module attached to a Vertex 70 spectrometer (Bruker Optik GmbH).

### DETAILED DESCRIPTION OF THE INVENTION

The present invention may be understood more readily by reference to the following detailed description of exemplary embodiments of the invention and the examples included therein.

It is to be understood that this invention is not limited to specific preparation methods that may of course vary. It is also to be understood that the terminology used herein is for the purpose of describing particular embodiments only and is not intended to be limiting.

Embodiment A1 of the present invention provides a crystalline form of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (e.g. Form 1). The crystalline form of the invention can be identified by its unique solid state signatures with respect to, for example, powder X-ray diffraction (PXRD) data, solid state Nuclear Magnetic Resonance (ssNMR) data (e.g. ¹³C ssNMR data, ¹⁵N ssNMR data, and/or ¹⁹F ssNMR), and/or FT-Raman Spectroscopy data provided herein.

Embodiment A2 is a further embodiment of Embodiment A1, wherein the crystalline form has a purity of greater than 90%.

Embodiment A3 is a further embodiment of Embodiment A1, wherein the crystalline form has a purity of greater than 95%.

Embodiment A4 is a further embodiment of Embodiment A1, wherein the crystalline form has a purity of greater than 97%.

Embodiment A5 is a further embodiment of Embodiment A1, wherein the crystalline form has a purity of greater than 99%.

Embodiment A6 is a further embodiment of any one of Embodiments A1 to A5, wherein the crystalline form is designated as Form 1. Form 1 of the invention can be identified by its unique solid state signatures with respect to, for example, powder X-ray diffraction (PXRD) data, solid state Nuclear Magnetic Resonance (ssNMR) data (e.g. ¹³C ssNMR data, ¹⁵N ssNMR data, and/or ¹⁹F ssNMR), and/or FT-Raman Spectroscopy data provided herein.

Embodiment A7 is a further embodiment of any one of Embodiments A1 to A6, wherein the crystalline form has a powder X-ray diffraction pattern (PXRD) comprising one peak, in terms of 2θ (Cu Kα radiation source, wavelength of 1.5406 Å), selected from those at 14.3±0.2°, 17.5±0.2°, and 18.0±0.2°.

Embodiment A8 is a further embodiment of any one of Embodiments A1 to A7, wherein the crystalline form has a powder X-ray diffraction pattern (PXRD) comprising one peak, in terms of 2θ, at 14.3±0.2°.

Embodiment A9 is a further embodiment of any one of Embodiments A1 to A8, wherein the crystalline form has a powder X-ray diffraction pattern (PXRD) comprising one peak, in terms of 2θ, at 17.5±0.2°.

Embodiment A10 is a further embodiment of any one of Embodiments A1 to A9, wherein the crystalline form has a powder X-ray diffraction pattern (PXRD) comprising one peak, in terms of 2θ, at 18.0±0.2°.

Embodiment A11 is a further embodiment of any one of Embodiments A1 to A6, wherein the crystalline form has a powder X-ray diffraction pattern (PXRD) comprising two peaks, in terms of 2θ, selected from those at 14.3±0.2°, 17.5±0.2°, and 18.0±0.2°.

Embodiment A12 is a further embodiment of any one of Embodiments A1 to A11, wherein the crystalline form has a powder X-ray diffraction pattern (PXRD) comprising peaks, in terms of 2θ, at 14.3±0.2°, 17.5±0.2°, and 18.0±0.2°.

Embodiment A13 is a further embodiment of any one of Embodiments A1 to A12, wherein the crystalline form has a powder X-ray diffraction pattern (PXRD) comprising peaks, in terms of 2θ, at 14.3±0.2°, 17.5±0.2°, 18.0±0.2°, and 23.4±0.2°.

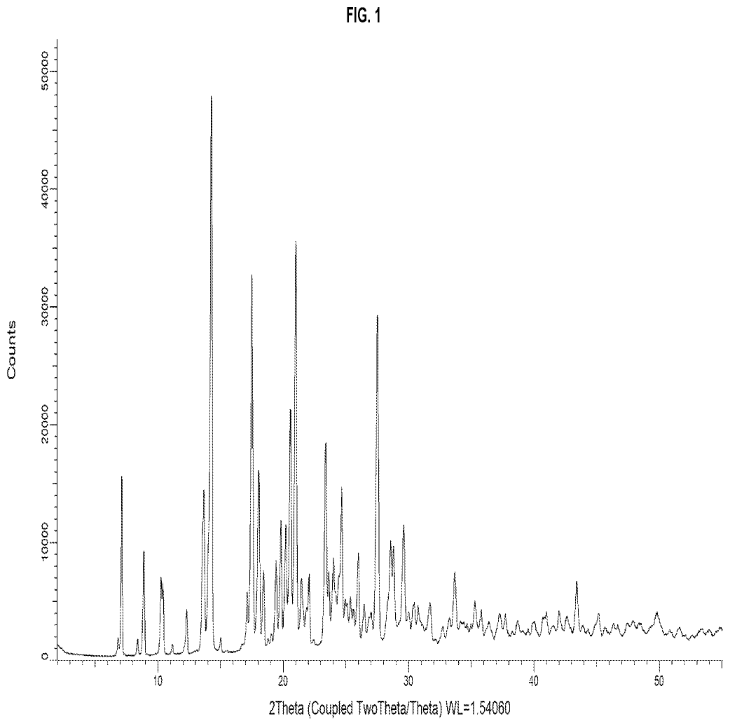

Embodiment A14 is a further embodiment of any one of Embodiments A1 to A13, wherein the crystalline form has a powder X-ray diffraction pattern (PXRD) comprising peaks, in terms of 2θ, at 14.3±0.2°, 17.5±0.2°, 18.0±0.2°, 23.4±0.2°, and 24.7 0.2°. In a further embodiment, the crystalline form has a powder X-ray diffraction pattern (PXRD) substantially the same as FIG. 1.

Embodiment A15 is a further embodiment of any one of Embodiments A1 to A14, wherein the crystalline form has a ¹³C ssNMR spectrum comprising one peak, in terms of chemical shifts, selected from those at 171.0±0.2 ppm and 141.3±0.2 ppm.

Embodiment A16 is a further embodiment of any one of Embodiments A1 to A15, wherein the crystalline form has a ¹³C ssNMR spectrum comprising one peak, in terms of chemical shifts, at 171.0±0.2 ppm.

Embodiment A17 is a further embodiment of any one of Embodiments A1 to A15, wherein the crystalline form has a ¹³C ssNMR spectrum comprising one peak, in terms of chemical shifts, at 141.3±0.2 ppm.

Embodiment A18 is a further embodiment of any one of Embodiments A1 to A17, wherein the crystalline form has a ¹³C ssNMR spectrum comprising peaks, in terms of chemical shifts, at 171.0±0.2 ppm and 141.3±0.2 ppm.

Embodiment A19 is a further embodiment of any one of Embodiments A1 to A18, wherein the crystalline form has a ¹³C ssNMR spectrum comprising peaks, in terms of chemical shifts, at 171.0±0.2 ppm, 141.3±0.2 ppm, and 64.0±0.2 ppm.

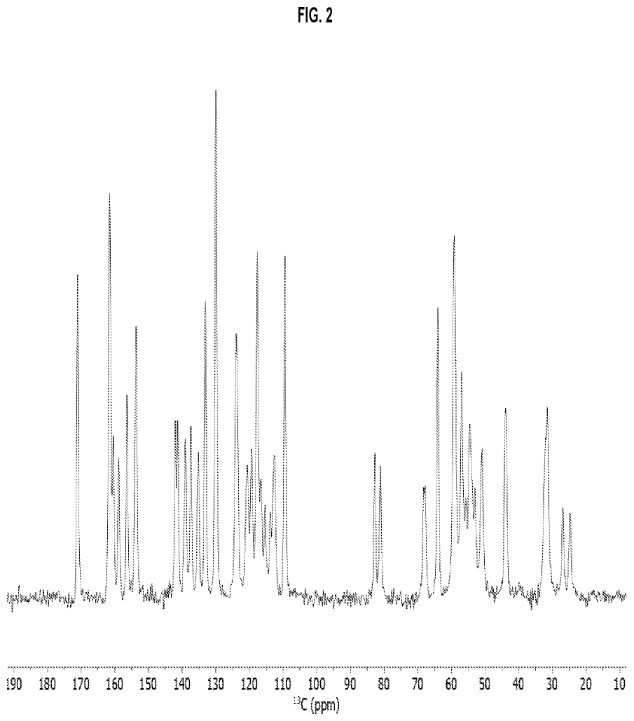

Embodiment A20 is a further embodiment of any one of Embodiments A1 to A19, wherein the crystalline form has a ¹³C ssNMR spectrum comprising peaks, in terms of chemical shifts, at 171.0±0.2 ppm, 141.9±0.2 ppm, 141.3±0.2 ppm, 120.7±0.2 ppm, and 64.0±0.2 ppm. In a further embodiment, the crystalline form has a ¹³C ssNMR spectrum substantially the same as FIG. 2.

Embodiment A21 is a further embodiment of any one of Embodiments A1 to A20, wherein the crystalline form has a ¹⁵N ssNMR spectrum comprising one peak, in terms of chemical shifts, at −339.9±0.2 ppm or −223.4±0.2 ppm.

Embodiment A22 is a further embodiment of any one of Embodiments A1 to A21, wherein the crystalline form has a ¹⁵N ssNMR spectrum comprising one peak, in terms of chemical shifts, at −339.9±0.2 ppm.

Embodiment A23 is a further embodiment of any one of Embodiments A1 to A22, wherein the crystalline form has a ¹⁵N ssNMR spectrum comprising one peak, in terms of chemical shifts, at −223.4±0.2 ppm.

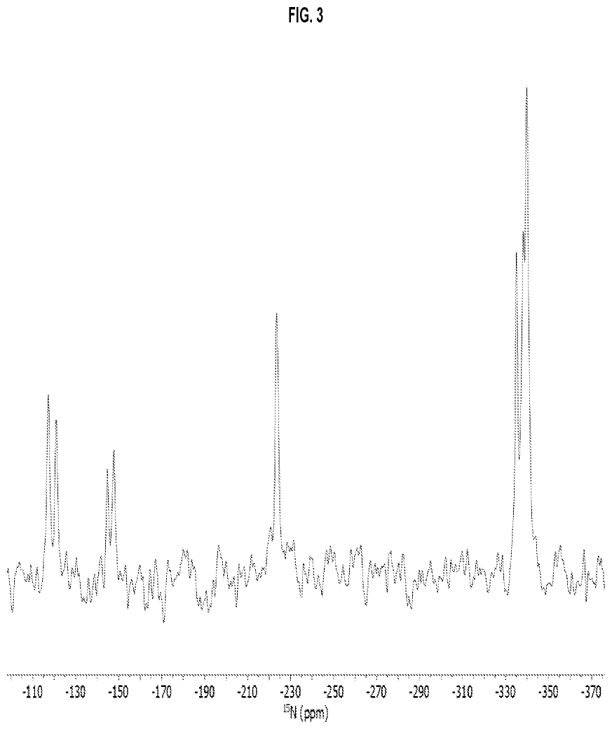

Embodiment A24 is a further embodiment of any one of Embodiments A1 to A23, wherein the crystalline form has a ¹⁵N ssNMR spectrum comprising peaks, in terms of chemical shifts, at −339.9±0.2 ppm and −223.4±0.2 ppm. In a further embodiment, the crystalline form has a ¹⁵N ssNMR spectrum substantially the same as FIG. 3.

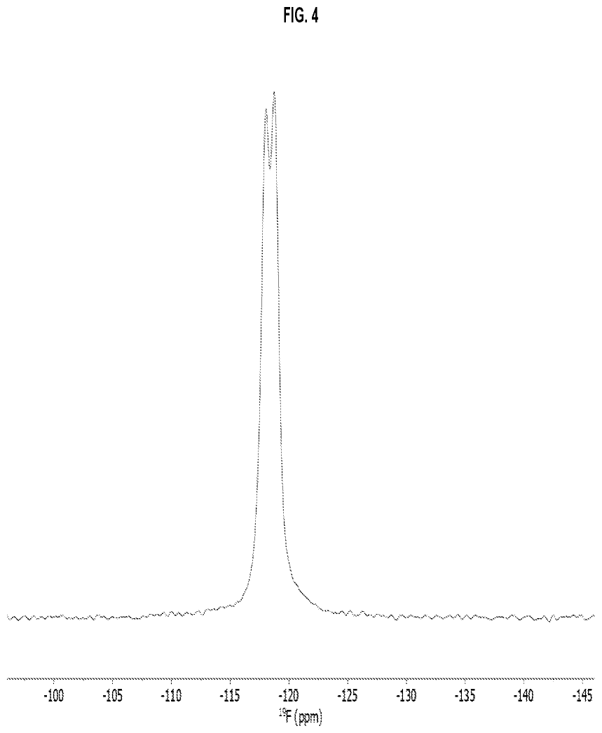

Embodiment A25 is a further embodiment of any one of Embodiments A1 to A24, wherein the crystalline form has a ¹⁹F ssNMR spectrum comprising one peak, in terms of chemical shifts, at −118.8±0.2 ppm. In a further embodiment, the crystalline form has a ¹⁹F ssNMR spectrum substantially the same as FIG. 4.

Embodiment A26 is a further embodiment of any one of Embodiments A1 to A25, wherein the crystalline form has an FT-Raman spectrum comprising one peak, in terms of wavenumbers (cm⁻¹), selected from those at 1371±2 cm⁻¹, 430±2 cm⁻¹, and 416±2 cm⁻¹.

Embodiment A27 is a further embodiment of any one of Embodiments A1 to A26, wherein the crystalline form has an FT-Raman spectrum comprising one peak, in terms of wavenumbers (cm⁻¹), at 1371±2 cm⁻¹.

Embodiment A28 is a further embodiment of any one of Embodiments A1 to A27, wherein the crystalline form has an FT-Raman spectrum comprising one peak, in terms of wavenumbers (cm⁻¹), at 430±2 cm⁻¹.

Embodiment A29 is a further embodiment of any one of Embodiments A1 to A28, wherein the crystalline form has an FT-Raman spectrum comprising one peak, in terms of wavenumbers (cm⁻¹), at 416±2 cm⁻¹.

Embodiment A30 is a further embodiment of any one of Embodiments A1 to A26, wherein the crystalline form has an FT-Raman spectrum comprising two peaks, in terms of wavenumbers (cm⁻¹), selected from those at 1371±2 cm⁻¹, 430±2 cm⁻¹, and 416±2 cm⁻¹.

Embodiment A31 is a further embodiment of Embodiment 30, wherein the crystalline form has an FT-Raman spectrum comprising two peaks, in terms of wavenumbers (cm⁻¹), at 1371±2 cm⁻¹ and 430±2 cm⁻¹.

Embodiment A32 is a further embodiment of Embodiment 30, wherein the crystalline form has an FT-Raman spectrum comprising two peaks, in terms of wavenumbers (cm⁻¹), at 1371±2 cm⁻¹ and 416±2 cm⁻¹.

Embodiment A33 is a further embodiment of Embodiment 30, wherein the crystalline form has an FT-Raman spectrum comprising two peaks, in terms of wavenumbers (cm⁻¹), at 430±2 cm⁻¹ and 416±2 cm⁻¹.

Embodiment A34 is a further embodiment of any one of Embodiments A1 to A33, wherein the crystalline form has an FT-Raman spectrum comprising peaks, in terms of wavenumbers (cm⁻¹), at 1371±2 cm⁻¹, 430±2 cm⁻¹, and 416±2 cm⁻¹.

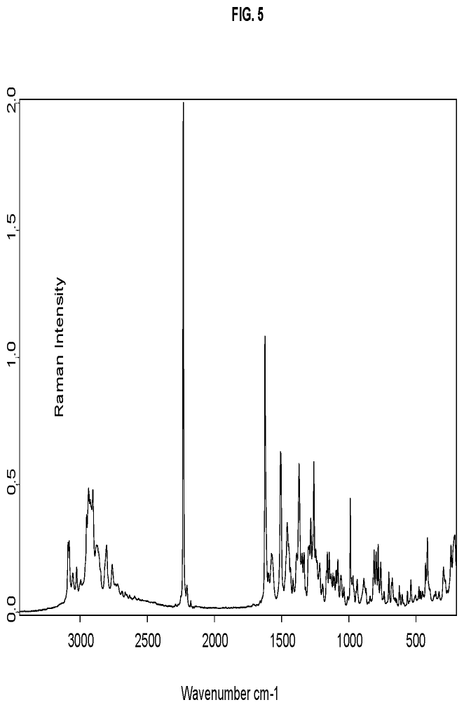

Embodiment A35 is a further embodiment of any one of Embodiments A1 to A34, wherein the crystalline form has a FT-Raman spectrum comprising peaks, in terms of wavenumbers (cm⁻¹), at 1371±2 cm⁻¹, 430±2 cm⁻¹, 416±2 cm⁻¹, and 3026±2 cm⁻¹. In a further embodiment, the crystalline form has an FT-Raman spectrum substantially the same as FIG. 5.

Embodiment A36 is a further embodiment of any one of Embodiments A1 to A35, wherein the crystalline form contains no more than about 1.0%, about 0.8%, about 0.7%, about 0.6%, about 0.5%, about 0.4%, or about 0.3% by weight of a compound of Formula IMP-1 or a salt thereof.

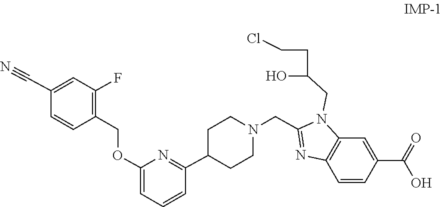 [SMILES: N#Cc1ccc(COc2cccc(C3CCN(Cc4nc5ccc(C(=O)O)cc5n4CC(O)CCCl)CC3)n2)c(F)c1]

Embodiment A37 is a further embodiment of any one of Embodiments A1 to A36, wherein the crystalline form contains no more than about 0.5%, about 0.4%, about 0.3%, about 0.2%, or about 0.1% by weight of a compound of Formula IMP-1 or a salt thereof.

Embodiment A38 is a further embodiment of any one of Embodiments A1 to A37, wherein the crystalline form contains no more than about 0.2%, about 0.1%, or about 0.05% by weight of a compound of Formula IMP-1 or a salt thereof.

Embodiment A39 is a further embodiment of any one of Embodiments A1 to A38, wherein the crystalline form contains no more than about 0.1% or about 0.05% by weight of a compound of Formula IMP-1 or a salt thereof.

Embodiment A40 is a further embodiment of any one of Embodiments A1 to A39, wherein the crystalline form contains no more than about 1.0%, about 0.8%, about 0.7%, about 0.6%, about 0.5%, about 0.4%, or about 0.3% by weight of a compound of Formula IMP-2 or a salt thereof.

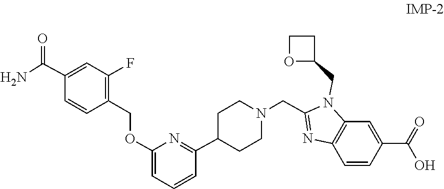 [SMILES: NC(=O)c1ccc(COc2cccc(C3CCN(Cc4nc5ccc(C(=O)O)cc5n4C[C@@H]4CCO4)CC3)n2)c(F)c1]

Embodiment A41 is a further embodiment of any one of Embodiments A1 to A40, wherein the crystalline form contains no more than about 0.5%, about 0.4%, about 0.3%, about 0.2%, or about 0.1% by weight of a compound of Formula IMP-2 or a salt thereof.

Embodiment A42 is a further embodiment of any one of Embodiments A1 to A41, wherein the crystalline form contains no more than about 0.2% or about 0.1% by weight of a compound of Formula IMP-2 or a salt thereof.

Embodiment A43 is a further embodiment of any one of Embodiments A1 to A42, wherein the crystalline form contains no more than about 0.1% or about 0.05% by weight of a compound of Formula IMP-2 or a salt thereof.

Embodiment A44 is a further embodiment of any one of Embodiments A1 to A43, wherein the crystalline form contains no more than about 1.0%, about 0.8%, about 0.7%, about 0.6%, about 0.5%, about 0.4%, or about 0.3% by weight of a compound of Formula IMP-3 or a salt thereof.

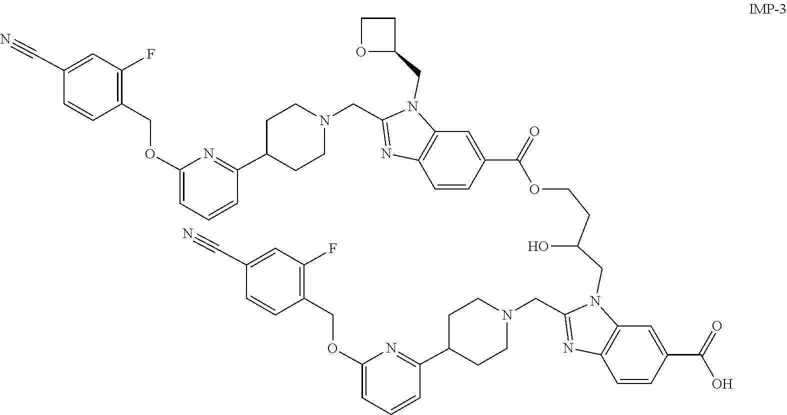 [SMILES: N#Cc1ccc(COc2cccc(C3CCN(Cc4nc5ccc(C(=O)O)cc5n4CC(O)CCOC(=O)c4ccc5nc(CN6CCC(c7cccc(OCc8ccc(C#N)cc8F)n7)CC6)n(C[C@@H]6CCO6)c5c4)CC3)n2)c(F)c1]

Embodiment A45 is a further embodiment of any one of Embodiments A1 to A44, wherein the crystalline form contains no more than about 0.5%, about 0.4%, about 0.3%, about 0.2%, or about 0.1% by weight of a compound of Formula IMP-3 or a salt thereof.

Embodiment A46 is a further embodiment of any one of Embodiments A1 to A45, wherein the crystalline form contains no more than about 0.2% or about 0.1% by weight of a compound of Formula IMP-3 or a salt thereof.

Embodiment A47 is a further embodiment of any one of Embodiments A1 to A46, wherein the crystalline form contains no more than about 0.1% or about 0.05% by weight of a compound of Formula IMP-3 or a salt thereof.

The present invention further provides a method for preparing Form 1 of tris salt of Compound 1 (e.g. any one of Embodiments A1 to A47) comprising precipitating (crystalizing) the Form 1 from a solution, wherein the solution comprises tris salt of Compound 1 and a solvent, and wherein the solvent comprises 1-propanol. In some further embodiments, the solvent comprises water and 1-propanol.

The present invention further provides a method for preparing Form 1 of tris salt of Compound 1 (e.g. any one of Embodiments A1 to A47) comprising precipitating (crystalizing) the Form 1 from a solution, wherein the solution comprises tris salt of Compound 1 and a mixed solvent, and wherein the mixed solvent comprises water and DMSO. In a further embodiment, the method further comprising adding seed crystals of Form 1 of tris salt of Compound 1 to the solution.

Precipitation (crystalizing) of Form 1 of tris salt of Compound 1 (e.g. any one of Embodiments A1 to A47) can be induced by any of the various well-known methods of precipitation (crystallization). For example, precipitation can be induced by cooling the solution or evaporation of solvents (optionally under reduced pressure). For another example, precipitation can be induced by scratching (bottom or side of the container/vessel). For yet another example, precipitation can be induced by adding seed crystals of Form 1 of tris salt of Compound 1.

Embodiment B1 of the present invention provides a pharmaceutical composition comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and a pharmaceutically acceptable carrier, wherein at least 5% of the tris salt of Compound 1 is present as the crystalline form of any one of Embodiments A1 to A47.

Embodiment B2 is a further embodiment of Embodiment B1 wherein at least 10% of the tris salt of Compound 1 is present as the crystalline form of any one of Embodiments A1 to A47.

Embodiment B3 is a further embodiment of Embodiment B1 wherein at least 20% of the tris salt of Compound 1 is present as the crystalline form of any one of Embodiments A1 to A47.

Embodiment B4 is a further embodiment of Embodiment B1 wherein at least 30% of the tris salt of Compound 1 is present as the crystalline form of any one of Embodiments A1 to A47.

Embodiment B5 is a further embodiment of Embodiment B1 wherein at least 40% of the tris salt of Compound 1 is present as the crystalline form of any one of Embodiments A1 to A47.

Embodiment B6 is a further embodiment of Embodiment B1 wherein at least 50% of the tris salt of Compound 1 is present as the crystalline form of any one of Embodiments A1 to A47.

Embodiment B7 is a further embodiment of Embodiment B1 wherein at least 60% of the tris salt of Compound 1 is present as the crystalline form of any one of Embodiments A1 to A47.

Embodiment B8 is a further embodiment of Embodiment B1 wherein at least 70% of the tris salt of Compound 1 is present as the crystalline form of any one of Embodiments A1 to A47.

Embodiment B9 is a further embodiment of Embodiment B1 wherein at least 80% of the tris salt of Compound 1 is present as the crystalline form of any one of Embodiments A1 to A47.

Embodiment B10 is a further embodiment of Embodiment B1 wherein at least 90% of the tris salt of Compound 1 is present as the crystalline form of any one of Embodiments A1 to A47.

Embodiment B11 is a further embodiment of Embodiment B1 wherein at least 95% of the tris salt of Compound 1 is present as the crystalline form of any one of Embodiments A1 to A47.

Embodiment B12 is a further embodiment of Embodiment B1 wherein at least 97% of the tris salt of Compound 1 is present as the crystalline form of any one of Embodiments A1 to A47.

Embodiment B13 is a further embodiment of Embodiment B1 wherein at least 99% of the tris salt of Compound 1 is present as the crystalline form of any one of Embodiments A1 to A47.

Embodiment C1 of the present invention provides an amorphous form of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt. The amorphous form of tris salt of Compound 1 does not give distinctive powder X-ray diffraction patterns (i.e., it PXRD does not have sharp peaks as in a PXRD for Form 1). The amorphous form of the invention can be identified by its unique solid state signatures with respect to, for example, powder X-ray diffraction (PXRD) data, solid state Nuclear Magnetic Resonance (ssNMR) data (e.g. ¹³C ssNMR data, ¹⁵N ssNMR data, and/or ¹⁹F ssNMR), and/or FT-Raman Spectroscopy data provided herein.

Embodiment C2 is a further embodiment of Embodiment C1 wherein the amorphous form is substantially pure.

Embodiment C3 is a further embodiment of Embodiment C1, wherein the amorphous form has a purity of greater than 90%.

Embodiment C4 is a further embodiment of Embodiment C1, wherein the amorphous form has a purity of greater than 95%.

Embodiment C5 is a further embodiment of Embodiment C1, wherein the amorphous form has a purity of greater than 97%.

Embodiment C6 is a further embodiment of Embodiment C1, wherein the amorphous form has a purity of greater than 99%.

Embodiment C7 is a further embodiment of any one of Embodiments C1 to C7, wherein the amorphous form is designated as Form 2, which can be identified by its unique solid state signatures with respect to, for example, powder X-ray diffraction (PXRD) data, solid state Nuclear Magnetic Resonance (ssNMR) data (e.g. ¹³C ssNMR data, ¹⁵N ssNMR data, and/or ¹⁹F ssNMR), and/or FT-Raman Spectroscopy data provided herein.

Embodiment C8 is a further embodiment of any one of Embodiments C1 to C7, wherein the amorphous form has a ¹³C ssNMR spectrum comprising one peak, in terms of chemical shifts, selected from those at 174.0±0.2 ppm, 143.9±0.3 ppm, and 62.2±0.3 ppm.

Embodiment C9 is a further embodiment of any one of Embodiments C1 to C8, wherein the amorphous form has a ¹³C ssNMR spectrum comprising one peak, in terms of chemical shifts, at 174.0±0.2 ppm.

Embodiment C10 is a further embodiment of any one of Embodiments C1 to C9, wherein the amorphous form has a ¹³C ssNMR spectrum comprising one peak, in terms of chemical shifts, at 143.9±0.3 ppm.

Embodiment C11 is a further embodiment of any one of Embodiments C1 to C10, wherein the amorphous form has a ¹³C ssNMR spectrum comprising one peak, in terms of chemical shifts, at 62.2±0.3 ppm.

Embodiment C12 is a further embodiment of any one of Embodiments C1 to C11, wherein the amorphous form has a ¹³C ssNMR spectrum comprising two peaks, in terms of chemical shifts, selected from those at 174.0±0.2 ppm, 143.9±0.3 ppm, and 62.2±0.3 ppm.

Embodiment C13 is a further embodiment of any one of Embodiments C1 to C12, wherein the amorphous form has a ¹³C ssNMR spectrum comprising two peaks, in terms of chemical shifts, at 174.0±0.2 ppm and 143.9±0.3 ppm.

Embodiment C14 is a further embodiment of any one of Embodiments C1 to C12, wherein the amorphous form has a ¹³C ssNMR spectrum comprising two peaks, in terms of chemical shifts, at 174.0±0.2 ppm and 62.2±0.3 ppm.

Embodiment C15 is a further embodiment of any one of Embodiments C1 to C12, has a ¹³C ssNMR spectrum comprising two peaks, in terms of chemical shifts, at 143.9±0.3 ppm and 62.2±0.3 ppm.

Embodiment C16 is a further embodiment of any one of Embodiments C1 to C12, wherein the amorphous form has a ¹³C ssNMR spectrum comprising peaks, in terms of chemical shifts, selected from those at 174.0±0.2 ppm, 143.9±0.3 ppm, and 62.2±0.3 ppm.

Embodiment C17 is a further embodiment of any one of Embodiments C1 to C16, wherein the amorphous form has a ¹³C ssNMR spectrum comprising peaks, in terms of chemical shifts, selected from those at 174.0±0.2 ppm, 143.9±0.3 ppm, 62.2±0.3 ppm, and 29.6±0.2 ppm.

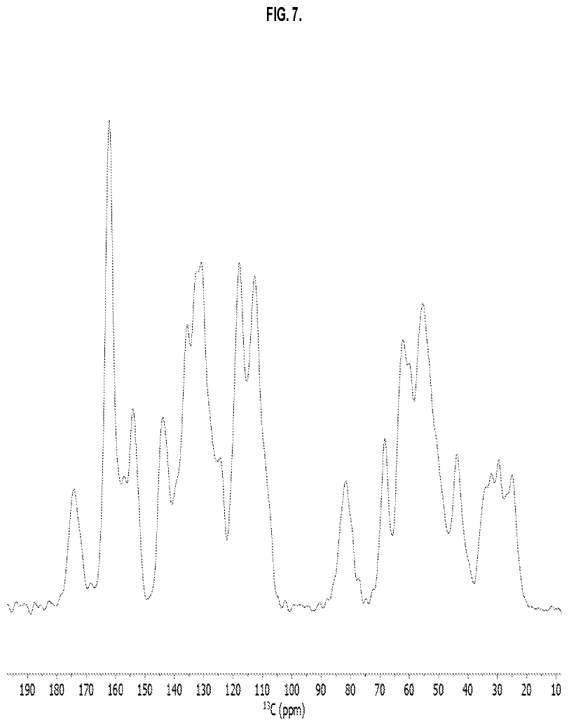

Embodiment C18 is a further embodiment of any one of Embodiments C1 to C17, wherein the amorphous form has a ¹³C ssNMR spectrum comprising peaks, in terms of chemical shifts, selected from those at 174.0±0.2 ppm, 143.9±0.3 ppm, 62.2±0.3 ppm, 29.6±0.2 ppm, and 130.8±0.3 ppm. In a further embodiment, the crystalline form has a ¹³C ssNMR spectrum substantially the same as FIG. 7.

Embodiment C19 is a further embodiment of any one of Embodiments C1 to C18, wherein the amorphous form has a ¹⁵N ssNMR spectrum comprising one peak, in terms of chemical shifts, at −332.7±0.8 ppm or −229±1.0 ppm.

Embodiment C20 is a further embodiment of any one of Embodiments C1 to C19, wherein the amorphous form has a ¹⁵N ssNMR spectrum comprising one peak, in terms of chemical shifts, at −332.7±0.8 ppm.

Embodiment C21 is a further embodiment of any one of Embodiments C1 to C19, wherein the amorphous form has a ¹⁵N ssNMR spectrum comprising one peak, in terms of chemical shifts, at −229±1.0 ppm.

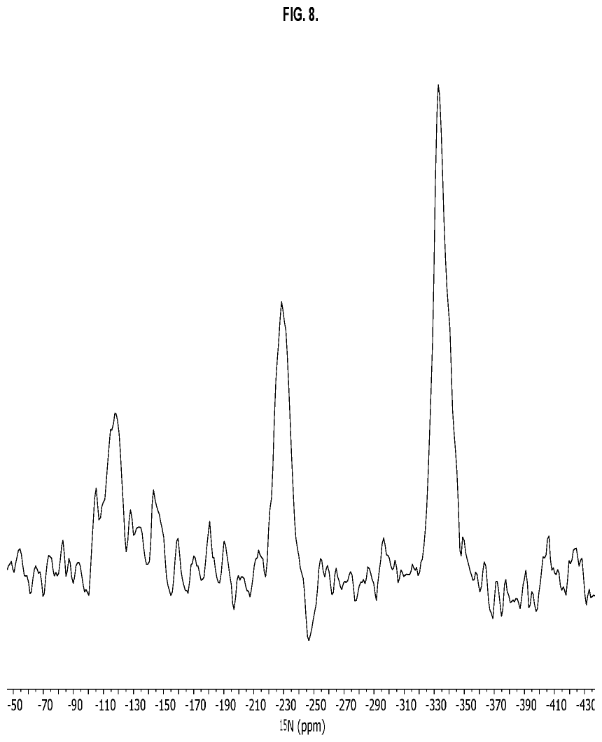

Embodiment C22 is a further embodiment of any one of Embodiments C1 to C19, wherein the amorphous form has a ¹⁵N ssNMR spectrum comprising peaks, in terms of chemical shifts, at −332.7±0.8 ppm and −229±1.0 ppm. In a further embodiment, the crystalline form has a ¹⁵N ssNMR spectrum substantially the same as FIG. 8.

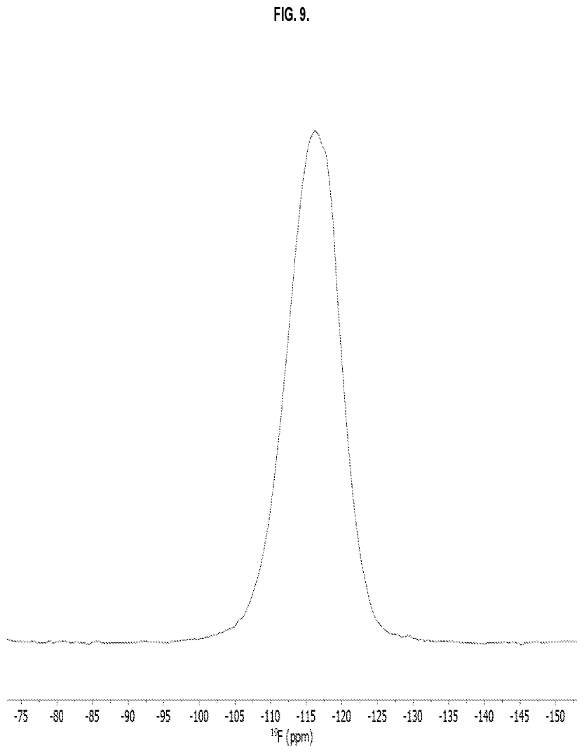

Embodiment C23 is a further embodiment of any one of Embodiments C1 to C22, wherein the amorphous form has a ¹⁹F ssNMR spectrum comprising one peak, in terms of chemical shifts, at −116.3±0.8 ppm. In a further embodiment, the crystalline form has a ¹⁹F ssNMR spectrum substantially the same as FIG. 9.

Embodiment C24 is a further embodiment of any one of Embodiments C1 to C23, wherein the amorphous form has an FT-Raman spectrum comprising one peak, in terms of wavenumbers (cm⁻¹), selected from those at 1513±2 cm⁻¹, 1278±2 cm⁻¹, and 1378±2 cm⁻¹.

Embodiment C25 is a further embodiment of any one of Embodiments C1 to C24, wherein the amorphous form has an FT-Raman spectrum comprising one peak, in terms of wavenumbers (cm⁻¹), at 1513±2 cm⁻¹.

Embodiment C26 is a further embodiment of any one of Embodiments C1 to C24, wherein the amorphous form has an FT-Raman spectrum comprising one peak, in terms of wavenumbers (cm⁻¹), at 1278±2 cm⁻¹.

Embodiment C27 is a further embodiment of any one of Embodiments C1 to C24, wherein the amorphous form has an FT-Raman spectrum comprising one peak, in terms of wavenumbers (cm⁻¹), at 1378±2 cm⁻¹.

Embodiment C28 is a further embodiment of any one of Embodiments C1 to C24, wherein the amorphous form has an FT-Raman spectrum comprising two peaks, in terms of wavenumbers (cm⁻¹), at 1513±2 cm⁻¹ and 1278±2 cm⁻¹.

Embodiment C29 is a further embodiment of any one of Embodiments C1 to C24, wherein the amorphous form has an FT-Raman spectrum comprising two peaks, in terms of wavenumbers (cm⁻¹), at 1513±2 cm⁻¹ and 1378±2 cm⁻¹.

Embodiment C30 is a further embodiment of any one of Embodiments C1 to C24, wherein the amorphous form has an FT-Raman spectrum comprising two peaks, in terms of wavenumbers (cm⁻¹), at 1278±2 cm⁻¹ and 1378±2 cm⁻¹.

Embodiment C31 is a further embodiment of any one of Embodiments C1 to C30, wherein the amorphous form has an FT-Raman spectrum comprising peaks, in terms of wavenumbers (cm⁻¹), at 1513±2 cm⁻¹, 1278±2 cm⁻¹, and 1378±2 cm⁻¹. In a further embodiment, the crystalline form has an FT-Raman spectrum substantially the same as FIG. 7.

Embodiment C32 is a further embodiment of any one of Embodiments C1 to C31, wherein the amorphous form contains no more than about 1.0%, about 0.8%, about 0.7%, about 0.6%, about 0.5%, about 0.4%, or about 0.3% by weight of a compound of Formula IMP-1 or a salt thereof.

Embodiment C33 is a further embodiment of any one of Embodiments C1 to C32, wherein the amorphous form contains no more than about 0.5%, about 0.4%, about 0.3%, about 0.2%, or about 0.1% by weight of a compound of Formula IMP-1 or a salt thereof.

Embodiment C34 is a further embodiment of any one of Embodiments C1 to C33, wherein the amorphous form contains no more than about 0.2%, about 0.1%, or about 0.05% by weight of a compound of Formula IMP-1 or a salt thereof.

Embodiment C35 is a further embodiment of any one of Embodiments C1 to C34, wherein the amorphous form contains no more than about 0.1% or about 0.05% by weight of a compound of Formula IMP-1 or a salt thereof.

Embodiment C36 is a further embodiment of any one of Embodiments C1 to C35, wherein the amorphous form contains no more than about 1.0%, about 0.8%, about 0.7%, about 0.6%, about 0.5%, about 0.4%, or about 0.3% by weight of a compound of Formula IMP-2 or a salt thereof.

Embodiment C37 is a further embodiment of any one of Embodiments C1 to C36, wherein the amorphous form contains no more than about 0.5%, about 0.4%, about 0.3%, about 0.2%, or about 0.1% by weight of a compound of Formula IMP-2 or a salt thereof.

Embodiment C38 is a further embodiment of any one of Embodiments C1 to C37, wherein the amorphous form contains no more than about 0.2% or about 0.1% by weight of a compound of Formula IMP-2 or a salt thereof.

Embodiment C39 is a further embodiment of any one of Embodiments C1 to C38, wherein the amorphous form contains no more than about 0.1% or about 0.05% by weight of a compound of Formula IMP-2 or a salt thereof.

Embodiment C40 is a further embodiment of any one of Embodiments C1 to C39, wherein the amorphous form contains no more than about 1.0%, about 0.8%, about 0.7%, about 0.6%, about 0.5%, about 0.4%, or about 0.3% by weight of a compound of Formula IMP-3 or a salt thereof.

Embodiment C41 is a further embodiment of any one of Embodiments C1 to C40, wherein the amorphous form contains no more than about 0.5%, about 0.4%, about 0.3%, about 0.2%, or about 0.1% by weight of a compound of Formula IMP-3 or a salt thereof.

Embodiment C42 is a further embodiment of any one of Embodiments C1 to C41, wherein the amorphous form contains no more than about 0.2% or about 0.1% by weight of a compound of Formula IMP-3 or a salt thereof.

Embodiment C43 is a further embodiment of any one of Embodiments C1 to C42, wherein the amorphous form contains no more than about 0.1% or about 0.05% by weight of a compound of Formula IMP-3 or a salt thereof.

Embodiment D1 of the present invention provides a pharmaceutical composition comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and a pharmaceutically acceptable carrier, wherein at least 5% of the tris salt of Compound 1 is present as the amorphous form of any one of Embodiments C1 to C43.

The present invention further provides a method for preparing Form 2 of tris salt of Compound 1 (e.g. any one of Embodiments C1 to C43) comprising ball milling Form 1 of tris salt of Compound 1 to provide Form 2 of tris salt of Compound 1.

Embodiment D2 is a further embodiment of Embodiment D1 wherein at least 10% of the tris salt of Compound 1 is present as the amorphous form of any one of Embodiments C1 to C43.

Embodiment D3 is a further embodiment of Embodiment D1 wherein at least 20% of the tris salt of Compound 1 is present as the amorphous form of any one of Embodiments C1 to C43.

Embodiment D4 is a further embodiment of Embodiment D1 wherein at least 30% of the tris salt of Compound 1 is present as the amorphous form of any one of Embodiments C1 to C43.

Embodiment D5 is a further embodiment of Embodiment D1 wherein at least 40% of the tris salt of Compound 1 is present as the amorphous form of any one of Embodiments C1 to C43.

Embodiment D6 is a further embodiment of Embodiment D1 wherein at least 50% of the tris salt of Compound 1 is present as the amorphous form of any one of Embodiments C1 to C43.

Embodiment D7 is a further embodiment of Embodiment D1 wherein at least 60% of the tris salt of Compound 1 is present as the amorphous form of any one of Embodiments C1 to C43.

Embodiment D8 is a further embodiment of Embodiment D1 wherein at least 70% of the tris salt of Compound 1 is present as the amorphous form of any one of Embodiments C1 to C43.

Embodiment D9 is a further embodiment of Embodiment D1 wherein at least 80% of the tris salt of Compound 1 is present as the amorphous form of any one of Embodiments C1 to C43.

Embodiment D10 is a further embodiment of Embodiment D1 wherein at least 90% of the tris salt of Compound 1 is present as the amorphous form of any one of Embodiments C1 to C43.

Embodiment D11 is a further embodiment of Embodiment D1 wherein at least 95% of the tris salt of Compound 1 is present as the amorphous form of any one of Embodiments C1 to C43.

Embodiment D12 is a further embodiment of Embodiment D1 wherein at least 97% of the tris salt of Compound 1 is present as the amorphous form of any one of Embodiments C1 to C43.

Embodiment D13 is a further embodiment of Embodiment D1 wherein at least 99% of the tris salt of Compound 1 is present as the amorphous form of any one of Embodiments C1 to C43.

Embodiment E1 of the present invention provides a pharmaceutical composition comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and a pharmaceutically acceptable carrier, wherein the tris salt of Compound 1 comprises the crystalline form of any one of Embodiments A1 to A47 and the amorphous form of any one of Embodiments C1 to C43.

Embodiment E2 is a further embodiment of Embodiment E1 wherein at least 1% of the tris salt of Compound 1 is present as the crystalline form of any one of Embodiments A1 to A47, and at least 1% of the tris salt of Compound 1 is present as the amorphous form of any one of Embodiments C1 to C43.

Embodiment E3 is a further embodiment of Embodiment E1 wherein at least 2% of the tris salt of Compound 1 is present as the crystalline form of any one of Embodiments A1 to A47, and at least 2% of the tris salt of Compound 1 is present as the amorphous form of any one of Embodiments C1 to C43.

Embodiment E4 is a further embodiment of Embodiment E1 wherein at least 5% of the tris salt of Compound 1 is present as the crystalline form of any one of Embodiments A1 to A47, and at least 5% of the tris salt of Compound 1 is present as the amorphous form of any one of Embodiments C1 to C43.

Embodiment E5 is a further embodiment of Embodiment E1 wherein at least 10% of the tris salt of Compound 1 is present as the crystalline form of any one of Embodiments A1 to A47, and at least 10% of the tris salt of Compound 1 is present as the amorphous form of any one of Embodiments C1 to C43.

Embodiment E6 is a further embodiment of Embodiment E1 wherein at least 10% of the tris salt of Compound 1 is present as the crystalline form of any one of Embodiments A1 to A47, and at least 5% of the tris salt of Compound 1 is present as the amorphous form of any one of Embodiments C1 to C43.

Embodiment F1 of the present invention provides a method for treating a disease or disorder comprising administering to a mammal in need of such treatment a therapeutically effective amount of the crystalline form of tris salt of Compound 1 of any one of Embodiments A1 to A47, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

Embodiment F2 is a further embodiment of Embodiment F1, wherein the disease or disorder is selected from obesity, NAFLD, NASH, NASH with fibrosis, T2D, and a cardiovascular disease.

Embodiment F3 is a further embodiment of Embodiment F1 or F2, wherein the crystalline form of tris salt of Compound 1 of any one of Embodiments A1 to A47 is administered in combination with an additional agent.

Embodiment F4 is a further embodiment of Embodiment F3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment F5 is a further embodiment of Embodiment F3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment F6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment F7 is a further embodiment of Embodiment F3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment F8 is a further embodiment of Embodiment F3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment F9 is a further embodiment of Embodiment F3, wherein the additional agent is 4-(4-(1-Isopropyl-7-oxo-1,4,6,7-tetrahydrospiro[indazole-5,4′-piperidine]-1′-carbonyl)-6-methoxypyridin-2-yl)benzoic acid, or a pharmaceutically acceptable salt thereof.

Embodiment F10 is a further embodiment of Embodiment F3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment F11 is a further embodiment of Embodiment F3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment F12 is a further embodiment of Embodiment F3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment F13 is a further embodiment of Embodiment F3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment G1 of the present invention provides use of the crystalline form of tris salt of Compound 1 of any one of Embodiments A1 to A47 for treating a disease or disorder as in Embodiment F1.

Embodiment G2 is a further embodiment of Embodiment G1, wherein the disease or disorder as in Embodiment F2.

Embodiment G3 is a further embodiment of Embodiment G1 or G2, wherein the crystalline form of tris salt of Compound 1 of any one of Embodiments A1 to A47 is used in combination with an additional agent.

Embodiment G4 is a further embodiment of Embodiment G3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment G5 is a further embodiment of Embodiment G3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment G6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment G7 is a further embodiment of Embodiment G3, wherein the additional agent is Form 1 of (S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment G8 is a further embodiment of Embodiment G3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment G9 is a further embodiment of Embodiment G3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment G10 is a further embodiment of Embodiment G3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment G11 is a further embodiment of Embodiment G3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment G12 is a further embodiment of Embodiment G3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment G13 is a further embodiment of Embodiment G3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment H1 of the present invention provides use of the crystalline form of tris salt of Compound 1 of any one of Embodiments A1 to A47 in manufacturing a medicament for treating a disease or disorder as in Embodiment F1.

Embodiment H2 is a further embodiment of Embodiment H1, wherein the disease or disorder as in Embodiment F2.

Embodiment H3 is a further embodiment of Embodiment H1 or H2, wherein the crystalline form of tris salt of Compound 1 of any one of Embodiments A1 to A47 is used in combination with an additional agent.

Embodiment H4 is a further embodiment of Embodiment H3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment H5 is a further embodiment of Embodiment H3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment H6 is a further embodiment of Embodiment H3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment H7 is a further embodiment of Embodiment H3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment H8 is a further embodiment of Embodiment H3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment H9 is a further embodiment of Embodiment H3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment H10 is a further embodiment of Embodiment H3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment H11 is a further embodiment of Embodiment H3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment H12 is a further embodiment of Embodiment H3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment H13 is a further embodiment of Embodiment H3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment J1 of the present invention provides the crystalline form of tris salt of Compound 1 of any one of Embodiments A1 to A47 for use in treating a disease or disorder as in Embodiment F1.

Embodiment J2 is a further embodiment of Embodiment J1, wherein the disease or disorder as in Embodiment F2.

Embodiment J3 is a further embodiment of Embodiment J1 or J2, wherein the crystalline form of tris salt of Compound 1 of any one of Embodiments A1 to A47 is used in combination with an additional agent.

Embodiment J4 is a further embodiment of Embodiment J3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment J5 is a further embodiment of Embodiment J3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment J6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment J7 is a further embodiment of Embodiment J3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment J8 is a further embodiment of Embodiment J3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment J9 is a further embodiment of Embodiment J3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment J10 is a further embodiment of Embodiment J3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment J11 is a further embodiment of Embodiment J3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment J12 is a further embodiment of Embodiment J3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment J13 is a further embodiment of Embodiment J3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment K1 of the present invention provides a method for treating a disease or disorder comprising administering to a mammal in need of such treatment a therapeutically effective amount of the amorphous form of tris salt of Compound 1 of Embodiments C1 to C43, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

Embodiment K2 is a further embodiment of Embodiment K1, wherein the disease or disorder is selected from obesity, NAFLD, NASH, NASH with fibrosis, T2D, and a cardiovascular disease.

Embodiment K3 is a further embodiment of Embodiment K1 or K2, wherein the amorphous form of tris salt of Compound 1 of any one of Embodiments C1 to C43 is used in combination with an additional agent.

Embodiment K4 is a further embodiment of Embodiment K3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment K5 is a further embodiment of Embodiment K3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment K6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment K7 is a further embodiment of Embodiment K3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment K8 is a further embodiment of Embodiment K3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment K9 is a further embodiment of Embodiment K3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment K10 is a further embodiment of Embodiment K3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment K11 is a further embodiment of Embodiment K3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment K12 is a further embodiment of Embodiment K3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment K13 is a further embodiment of Embodiment K3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment L1 of the present invention provides use of the amorphous form of tris salt of Compound 1 of Embodiments C1 to C43 for treating a disease or disorder as in Embodiment K1.

Embodiment L2 is a further embodiment of Embodiment L1, wherein the disease or disorder is the same as in Embodiment K2.

Embodiment L3 is a further embodiment of Embodiment L1 or L2, wherein the amorphous form of tris salt of Compound 1 of any one of Embodiments C1 to C43 is used in combination with an additional agent.

Embodiment L4 is a further embodiment of Embodiment L3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment L5 is a further embodiment of Embodiment L3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment L6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment L7 is a further embodiment of Embodiment L3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment L8 is a further embodiment of Embodiment L3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment L9 is a further embodiment of Embodiment L3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment L10 is a further embodiment of Embodiment L3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment L11 is a further embodiment of Embodiment L3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment L12 is a further embodiment of Embodiment L3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment L13 is a further embodiment of Embodiment L3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment M1 of the present invention provides use of the amorphous form of tris salt of Compound 1 of Embodiments C1 to C43 in manufacturing a medicament for treating a disease or disorder as in Embodiment K1.

Embodiment M2 is a further embodiment of Embodiment M1, wherein the disease or disorder is same as in Embodiment K2.

Embodiment M3 is a further embodiment of Embodiment M1 or M2, wherein the amorphous form of tris salt of Compound 1 of any one of Embodiments C1 to C43 is used in combination with an additional agent.

Embodiment M4 is a further embodiment of Embodiment M3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment M5 is a further embodiment of Embodiment M3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment M6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment M7 is a further embodiment of Embodiment M3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment M8 is a further embodiment of Embodiment M3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment M9 is a further embodiment of Embodiment M3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment M10 is a further embodiment of Embodiment M3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment M11 is a further embodiment of Embodiment M3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment M12 is a further embodiment of Embodiment M3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment M13 is a further embodiment of Embodiment M3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment N1 of the present invention provides the amorphous form of tris salt of Compound 1 of Embodiments C1 to C43 for use in treating a disease or disorder as in Embodiment K1.

Embodiment N2 is a further embodiment of Embodiment N1, wherein the disease or disorder is the same as in Embodiment F2.

Embodiment N3 is a further embodiment of Embodiment N1 or N2, wherein the amorphous form of tris salt of Compound 1 of any one of Embodiments C1 to C43 is used in combination with an additional agent.

Embodiment N4 is a further embodiment of Embodiment N3, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment N5 is a further embodiment of Embodiment N3, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment N6 is a further embodiment of Embodiment F3, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment N7 is a further embodiment of Embodiment N3, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment N8 is a further embodiment of Embodiment N3, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment N9 is a further embodiment of Embodiment N3, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment N10 is a further embodiment of Embodiment N3, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment N11 is a further embodiment of Embodiment N3, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment N12 is a further embodiment of Embodiment N3, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment N13 is a further embodiment of Embodiment N3, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment P1 of the present invention provides a pharmaceutical composition of any one of Embodiments B1 to B13 for use in treating a disease or disorder as in Embodiment K1.

Embodiment P2 is a further embodiment of Embodiment P1, wherein the disease or disorder is the same as in Embodiment K2.

Embodiment P3 of the present invention provides use of a pharmaceutical composition of any one of Embodiments B1 to B13 in treating a disease or disorder as in Embodiment K1.

Embodiment P4 is a further embodiment of Embodiment P3, wherein the disease or disorder is the same as in Embodiment K2.

Embodiment P5 of the present invention provides use of a pharmaceutical composition of any one of Embodiments B1 to B13 in manufacturing a medicament for treating a disease or disorder as in Embodiment K1. Embodiment P6 is a further embodiment of Embodiment P5, wherein the disease or disorder is the as in Embodiment K2.

Embodiment P7 of the present invention provides a method for treating a disease or disorder comprising administering to a mammal in need of such treatment a therapeutically effective amount of a pharmaceutical composition of any one of Embodiments B1 to B13, wherein the disease or disorder is the same as in Embodiment K1. Embodiment P8 is a further embodiment of Embodiment P7, wherein the disease or disorder is the same as in Embodiment K2.

Embodiment Q1 of the present invention provides a pharmaceutical composition of any one of Embodiments D1 to D13 for use in treating a disease or disorder as in Embodiment K1.

Embodiment Q3 of the present invention provides use of a pharmaceutical composition of any one of Embodiments D1 to D13 in treating a disease or disorder as in Embodiment K1. Embodiment Q4 is a further embodiment of Embodiment Q3, wherein the disease or disorder is the same as in Embodiment K2.

Embodiment Q5 of the present invention provides use of a pharmaceutical composition of any one of Embodiments D1 to D13 in manufacturing a medicament for treating a disease or disorder as in Embodiment K1. Embodiment Q6 is a further embodiment of Embodiment Q5, wherein the disease or disorder is the as in Embodiment K2.

Embodiment Q7 of the present invention provides a method for treating a disease or disorder comprising administering to a mammal in need of such treatment a therapeutically effective amount of a pharmaceutical composition of any one of Embodiments D1 to D13, wherein the disease or disorder is the same as in Embodiment K1. Embodiment Q8 is a further embodiment of Embodiment Q7, wherein the disease or disorder is the same as in Embodiment K2.

Embodiment R1 of the present invention provides a pharmaceutical composition of any one of Embodiments E1 to E5 for use in treating a disease or disorder as in Embodiment K1.

Embodiment R3 of the present invention provides use of a pharmaceutical composition of any one of Embodiments E1 to E5 in treating a disease or disorder as in Embodiment K1.

Embodiment R4 is a further embodiment of Embodiment R2, wherein the disease or disorder is the same as in Embodiment K2.

Embodiment R5 of the present invention provides use of a pharmaceutical composition of any one of Embodiments E1 to E5 in manufacturing a medicament for treating a disease or disorder as in Embodiment K1.

Embodiment R6 is a further embodiment of Embodiment R5, wherein the disease or disorder is the as in Embodiment K2.

Embodiment R7 of the present invention provides a method for treating a disease or disorder comprising administering to a mammal in need of such treatment a therapeutically effective amount of a pharmaceutical composition of any one of Embodiments E1 to E5, wherein the disease or disorder is the same as in Embodiment K1.

Embodiment R8 is a further embodiment of Embodiment R7, wherein the disease or disorder is the same as in Embodiment K2.

Any solid form of the present invention can be substantially pure. As used herein, the term “substantially pure” with reference to a particular solid form (e.g. a crystalline form) means that the particular solid form (e.g. the crystalline form) includes less than 15%, less than 10%, less than 5%, less than 3%, or less than 1% by weight of any other physical form of tris salt of Compound 1.

The term “substantially the same” when used to describe X-ray powder diffraction patterns is meant to include patterns in which peaks (in terms of 20) are within the deviations specified herein.

The term “substantially the same” when used to describe an ssNMR spectrum meant to include ssNMR spectra in which peaks (in terms of chemical shifts) are within the deviations specified herein.

The term “substantially the same” when used to describe an FT-Raman spectrum meant to include FT-Raman spectra in which peaks (in terms of wavenumber) are within the deviations specified herein.

The term “about” generally means within 10%, preferably within 5%, and more preferably within 1% of a given value or range. Alternatively, the term “about” means within an acceptable standard error of the mean, when considered by one skilled in the art.

The term “tris” means 1,3-dihydroxy-2-(hydroxymethyl) propan-2-amine, also known as THAM, tromethamine, or 2-amino-2-(hydroxymethyl) propane-1,3-diol.

Tris salt of Compound 1 means a salt of Compound 1 made using 1,3-dihydroxy-2-(hydroxymethyl) propan-2-amine and Compound 1. The tris is associated with the carboxylic acid moiety of Compound 1. Unless otherwise stated, when referencing the tris salt of Compound 1, the counterion and Compound 1 are in a stoichiometric ratio of about 1:1 (i.e. from 0.9:1.0 to 1.0:0.9, for example, from 0.95:1.00 to 1.00:0.95). Another chemical name for tris salt of Compound 1 is 1,3-dihydroxy-2-(hydroxymethyl) propan-2-aminium 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylate, which can also be represented, for example, by one of the following structures.

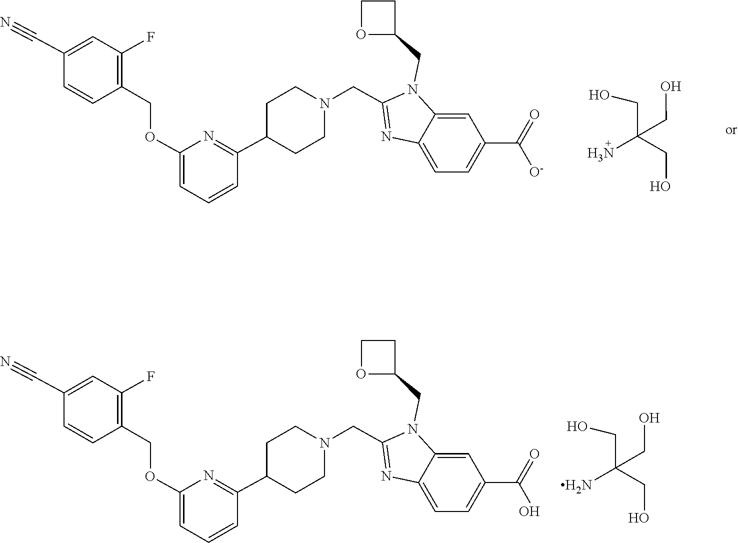

Those skilled in the art would readily understand that multiple nomenclatures can be used to name a same compound (including a same salt).

As used herein, the term “a disease or disorder modulated by GLP-1R” refers to a disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, a cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

Every example or embodiment of solid forms of the invention may be claimed individually or grouped together in any combination with any number of each and every embodiment described herein.

Room temperature (RT) or ambient temperature: 15 to 25° C.

Dimethyl sulfoxide: DMSO.

¹H Nuclear magnetic resonance (NMR) spectra were in all cases consistent with the proposed structures. Characteristic chemical shifts (8) are given in parts-per-million relative to the residual proton signal in the deuterated solvent (CHCl₃ at 7.27 ppm; CD₂HOD at 3.31 ppm; MeCN at 1.94 ppm; DMSO at 2.50 ppm) and are reported using conventional abbreviations for designation of major peaks: e.g. s, singlet; d, doublet; t, triplet; q, quartet; m, multiplet; br, broad. The symbol \hat{ }denotes that the 1H NMR peak area was assumed because the peak was partially obscured by water peak. The symbol \hat{ }\hat{ } denotes that the 1H NMR peak area was assumed because the peak was partially obscured by solvent peak.

The compounds and intermediates described below were named using the naming convention provided with ACD/ChemSketch 2012, ChemDraw, File Version C10H41, Build 69045 (Advanced Chemistry Development, Inc., Toronto, Ontario, Canada). The naming convention provided with ACD/ChemSketch 2012 is well known by those skilled in the art and it is believed that the naming convention provided with ACD/ChemSketch 2012 generally comports with the IUPAC (International Union for Pure and Applied Chemistry) recommendations on Nomenclature of Organic Chemistry and the CAS Index rules. One will note that the chemical names may have only parentheses or may have parentheses and brackets. The stereochemical descriptors may also be placed at different locations within the name itself, depending on the naming convention. One of ordinary skill in the art will recognize these formatting variations and understand they provide the same chemical structure.

Pharmaceutically acceptable salts include acid addition and base salts.

Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-Amino-2-(hydroxymethyl) propane-1,3-diol (tris or tromethamine) and zinc salts.

Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts may be prepared by one or more of three methods:
(i) by reacting a compound with the desired acid or base;
(ii) by removing an acid- or base-labile protecting group from a suitable precursor of a compound or by ring-opening a suitable cyclic precursor, for example, a lactone or lactam, using the desired acid or base; or
(iii) by converting one salt of a compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

Compounds and pharmaceutically acceptable salts, may exist in unsolvated and solvated forms. The term ‘solvate’ is used herein to describe a molecular complex comprising a compound or its salt, and one or more pharmaceutically acceptable solvent molecules, for example, ethanol. The term ‘hydrate’ is employed when said solvent is water. Form 1 and Form 2 described herein are believed to be unsolvated (and thus anhydrous).

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

A compound may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺ (CH₃)₃) polar head group. For more information, see Crystals and the Polarizing Microscope by N. H. Hartshorne and A. Stuart, 4th Edition (Edward Arnold, 1970).

Some compounds may exhibit polymorphism and/or one or more kinds of isomerism (e.g. optical, geometric or tautomeric isomerism). The solid forms (e.g. crystalline and/or amorphous forms) of the invention may also be isotopically labelled. Such variation is implicit to Compound 1 or its salt defined as they are by reference to their structural features and therefore within the scope of the invention.

Compounds containing one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds containing, for example, an imino, keto, or oxime group, or so-called valence tautomerism in compounds which contain an aromatic moiety. It follows that a single compound may exhibit more than one type of isomerism.

Certain pharmaceutically acceptable salts of Compound 1 may also contain a counterion which is optically active (e.g. d-lactate or l-lysine) or racemic (e.g. dl-tartrate or dl-arginine).

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, a racemic precursor containing a chiral ester may be separated by enzymatic resolution (see, for example, Int J Mol Sci 29682-29716 by A. C. L. M. Carvaho et. al. (2015)). In the case where a compound contains an acidic or basic moiety, a salt may be formed with an optically pure base or acid such as 1-phenylethylamine or tartaric acid. The resulting diastereomeric mixture may be separated by fractional crystallization and one or both of the diastereomeric salts converted to the corresponding pure enantiomer(s) by means well known to a skilled person. Alternatively, the racemate (or a racemic precursor) may be covalently reacted with a suitable optically active compound, for example, an alcohol, amine or benzylic chloride. The resulting diastereomeric mixture may be separated by chromatography and/or fractional crystallization by means well known to a skilled person to give the separated diastereomers as single enantiomers with 2 or more chiral centers. Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (SFC with Packed Columns), pp. 223-249 and references cited therein). In some relevant examples herein, columns were obtained from Chiral Technologies, Inc, West Chester, Pennsylvania, USA, a subsidiary of Daicel® Chemical Industries, Ltd., Tokyo, Japan.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Stereochemistry of Organic Compounds by E. L. Eliel and S. H. Wilen (Wiley, 1994).

It must be emphasised that Compound 1 and its salts have been drawn herein in a single tautomeric form, all possible tautomeric forms are included within the scope of the invention.

The present invention includes all pharmaceutically acceptable isotopically-labeled Compound 1 or a salt thereof wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, nitrogen, such as ¹³N and ¹⁵N, and oxygen, such as ¹⁵O, ¹⁷O and ¹⁸O.

Certain isotopically-labelled Compound 1 or a salt thereof, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

### Administration and Dosing

Typically, a compound (such as a crystalline form or an amorphous form) of the invention is administered in an amount effective to treat a condition as described herein. The compounds of the invention can be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, or topically.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention and/or compositions containing said compounds is based on a variety of factors, including the type, age, weight, sex and medical condition of the patient; the severity of the condition; the route of administration; and the activity of the particular compound employed. Thus the dosage regimen may vary widely. In one embodiment, the total daily dose of a compound of the invention is typically from about 0.001 to about 100 mg/kg (i.e., mg compound of the invention per kg body weight) for the treatment of the indicated conditions discussed herein. In another embodiment, total daily dose of the compound of the invention is from about 0.01 to about 30 mg/kg, and in another embodiment, from about 0.03 to about 10 mg/kg, and in yet another embodiment, from about 0.1 to about 3. It is not uncommon that the administration of the compounds of the invention will be repeated a plurality of times in a day (typically no greater than 4 times). Multiple doses per day typically may be used to increase the total daily dose, if desired.

For oral administration, the compositions may be provided in the form of tablets containing 0.1, 0.5, 1.0, 2.5, 5.0, 10.0, 15.0, 25.0, 30.0 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Suitable subjects according to the invention include mammalian subjects. In one embodiment, humans are suitable subjects. Human subjects may be of either gender and at any stage of development.

### Pharmaceutical Compositions

In another embodiment, the invention comprises pharmaceutical compositions. Such pharmaceutical compositions comprise a compound of the invention presented with a pharmaceutically acceptable carrier. Other pharmacologically active substances can also be present. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Pharmaceutically acceptable substances such as wetting agents or minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the antibody is administered by intravenous infusion or injection. In yet another embodiment, the antibody is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dose form is sub-lingual, such as, for example, a lozenge. In such solid dosage forms, the compounds of the invention are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the invention comprises a parenteral dose form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated-see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

### Co-Administration and Combination Therapy

The compounds (e.g. crystalline form or amorphous form) of the invention can be used alone, or in combination with other therapeutic agents. The invention provides any of the uses, methods or compositions as defined herein wherein the compound of any embodiment herein, or pharmaceutically acceptable salt thereof, or pharmaceutically acceptable solvate of said compound or salt, is used in combination with one or more other therapeutic agent discussed herein. This would include a pharmaceutical combination (e.g. a pharmaceutical composition) for the treatment of a disease or condition for which an agonist of the GLP-1R is indicated, comprising a crystalline form and/or an amorphous form of the invention, as defined in any of the embodiments described herein, and one or more other therapeutic agent discussed herein.

The administration of two or more compounds “in combination” means that all of the compounds are administered closely enough in time that each may generate a biological effect in the same time frame. The presence of one agent may alter the biological effects of the other compound(s). The two or more compounds may be administered simultaneously, concurrently sequentially, or separately (optionally with different dosing cycles). Additionally, simultaneous administration may be carried out by mixing the compounds prior to administration (or using a fixed dose combination) or by administering the compounds at the same point in time but as separate dosage forms (and optionally by different administration routes) at the same or different site of administration.

The phrases “concurrent administration,” “co-administration,” “simultaneous administration,” and “administered simultaneously” mean that the compounds are administered in combination.

In another embodiment, the invention provides methods of treatment that include administering compounds of the present invention in combination with one or more other pharmaceutical agents, wherein the one or more other pharmaceutical agents may be selected from the agents discussed herein.

In one embodiment, the compounds (e.g. crystalline form or amorphous form) of this invention are administered with an anti-diabetic agent including but not limited to a biguanide (e.g., metformin), a sulfonylurea (e.g., tolbutamide, glibenclamide, gliclazide, chlorpropamide, tolazamide, acetohexamide, glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In another embodiment, the compounds of this invention are administered with an anti-obesity agent including but not limited to peptide YY or an analogue thereof, a neuropeptide Y receptor type 2 (NPYR2) agonist, a NPYR1 or NPYR5 antagonist, a cannabinoid receptor type 1 (CB1R) antagonist, a lipase inhibitor (e.g., orlistat), a human proislet peptide (HIP), a melanocortin receptor 4 agonist (e.g., setmelanotide), a melanin concentrating hormone receptor 1 antagonist, a farnesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues thereof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In another embodiment, the compounds of this invention are administered in combination with one or more of the following: an agent to treat NASH including but not limited to PF-05221304, an FXR agonist (e.g., obeticholic acid), a PPAR a/8 agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor 21 (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976 or PF-05221304), a ketohexokinase (KHK) inhibitor, a diacylglyceryl acyltransferase 2 (DGAT2) inhibitor, a CB1 receptor antagonist, an anti-CB1R antibody, or an apoptosis signal-regulating kinase 1 (ASK1) inhibitor, including the pharmaceutically acceptable salts of the specifically named agents and the pharmaceutically acceptable solvates of said agents and salts.

Some specific compounds that can be used in combination with the compounds of the present invention for treating diseases or disorders described herein (e.g. NASH) include:
4-(4-(1-Isopropyl-7-oxo-1,4,6,7-tetrahydrospiro [indazole-5,4′-piperidine]-1′-carbonyl)-6-methoxypyridin-2-yl)benzoic acid, which is an example of a selective ACC inhibitor and was prepared as the free acid in Example 9 of U.S. Pat. No. 8,859,577, which is the U.S. national phase of International Application No. PCT/IB2011/054119, the disclosures of which are hereby incorporated herein by reference in their entireties for all purposes. Crystal forms of 4-(4-(1-Isopropyl-7-oxo-1,4,6,7-tetrahydrospiro [indazole-5,4′-piperidine]-1′-carbonyl)-6-methoxypyridin-2-yl)benzoic acid, including an anhydrous mono-tris form (Form 1) and a trihydrate of the mono-tris salt (Form 2), are described in International PCT Application No. PCT/IB2018/058966, the disclosure of which is hereby incorporated herein by reference in its entirety for all purposes;
(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof, and its crystalline solid forms (Form 1 and Form 2) is an example of a DGAT2 inhibitor described in Example 1 of U.S. Pat. No. 10,071,992, the disclosure of which is hereby incorporated herein by reference in its entirety for all purposes;
[(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid, or a pharmaceutically acceptable salt thereof, (including a crystalline free acid form thereof) is an example of a ketohexokinase inhibitor and is described in Example 4 of U.S. Pat. No. 9,809,579, the disclosure of which is hereby incorporated herein by reference in its entirety for all purposes; and
the FXR agonist Tropifexor or a pharmaceutically acceptable salt thereof is described in Example 1-1B of U.S. Pat. No. 9,150,568, the disclosure of which is hereby incorporated herein by reference in its entirety for all purposes.

These agents and compounds of the invention can be combined with pharmaceutically acceptable vehicles such as saline, Ringer's solution, dextrose solution, and the like. The particular dosage regimen, i.e., dose, timing and repetition, will depend on the particular individual and that individual's medical history.

Embodiment S1 of the present invention provides a pharmaceutical combination comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and an additional agent, wherein at least 5% of the tris salt of Compound 1 is present as the crystalline form of any one of Embodiments A1 to A47. In some further embodiments, the additional agent is a DGAT2 inhibitor. In other further embodiments, the additional agent is a selective ACC inhibitor.

Embodiment S2 is a further embodiment of Embodiment S1 wherein at least 10% of the tris salt of Compound 1 is present as the crystalline form of any one of Embodiments A1 to A47.

Embodiment S3 is a further embodiment of Embodiment S1 wherein at least 20% of the tris salt of Compound 1 is present as the crystalline form of any one of Embodiments A1 to A47.

Embodiment S4 is a further embodiment of Embodiment S1 wherein at least 30% of the tris salt of Compound 1 is present as the crystalline form of any one of Embodiments A1 to A47.

Embodiment S5 is a further embodiment of Embodiment S1 wherein at least 40% of the tris salt of Compound 1 is present as the crystalline form of any one of Embodiments A1 to A47.

Embodiment S6 is a further embodiment of Embodiment S1 wherein at least 50% of the tris salt of Compound 1 is present as the crystalline form of any one of Embodiments A1 to A47.

Embodiment S7 is a further embodiment of Embodiment S1 wherein at least 60% of the tris salt of Compound 1 is present as the crystalline form of any one of Embodiments A1 to A47.

Embodiment S8 is a further embodiment of Embodiment S1 wherein at least 70% of the tris salt of Compound 1 is present as the crystalline form of any one of Embodiments A1 to A47.

Embodiment S9 is a further embodiment of Embodiment S1 wherein at least 80% of the tris salt of Compound 1 is present as the crystalline form of any one of Embodiments A1 to A47.

Embodiment S10 is a further embodiment of Embodiment S1 wherein at least 90% of the tris salt of Compound 1 is present as the crystalline form of any one of Embodiments A1 to A47.

Embodiment S11 is a further embodiment of Embodiment S1 wherein at least 95% of the tris salt of Compound 1 is present as the crystalline form of any one of Embodiments A1 to A47.

Embodiment S12 is a further embodiment of Embodiment S1 wherein at least 97% of the tris salt of Compound 1 is present as the crystalline form of any one of Embodiments A1 to A47.

Embodiment S13 is a further embodiment of Embodiment S1 wherein at least 99% of the tris salt of Compound 1 is present as the crystalline form of any one of Embodiments A1 to A47.

Embodiment S14 is a further embodiment of any one of Embodiments S1 to S13, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment S15 is a further embodiment of any one of Embodiments S1 to S13, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment S16 is a further embodiment of any one of Embodiments S1 to S13, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment S17 is a further embodiment of any one of Embodiments S1 to S13, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment S18 is a further embodiment of any one of Embodiments S1 to S13, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment S19 is a further embodiment of any one of Embodiments S1 to S13, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment S20 is a further embodiment of any one of Embodiments S1 to S13, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment S21 is a further embodiment of any one of Embodiments S1 to S13, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment S22 is a further embodiment of any one of Embodiments S1 to S13, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment S23 is a further embodiment of any one of Embodiments S1 to S13, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment S24 is a further embodiment of any one of Embodiments S1 to S23, wherein the pharmaceutical combination comprising the tris salt of Compound 1 and the additional agent is a fixed-dose combination.

Embodiment S25 is a further embodiment of any one of Embodiments S1 to S23, wherein the pharmaceutical combination comprising the tris salt of Compound 1 and the additional agent is not a fixed-dose combination.

Embodiment T1 of the present invention provides a pharmaceutical combination comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and an additional agent, wherein at least 5% of the tris salt of Compound 1 is present as the amorphous form of any one of Embodiments C1 to C43.

Embodiment T2 is a further embodiment of Embodiment T1 wherein at least 10% of the tris salt of Compound 1 is present as the crystalline form of any one of Embodiments A1 to A47.

Embodiment T3 is a further embodiment of Embodiment T1 wherein at least 20% of the tris salt of Compound 1 is present as the crystalline form of any one of Embodiments A1 to A47.

Embodiment T4 is a further embodiment of Embodiment T1 wherein at least 30% of the tris salt of Compound 1 is present as the crystalline form of any one of Embodiments A1 to A47.

Embodiment T5 is a further embodiment of Embodiment T1 wherein at least 40% of the tris salt of Compound 1 is present as the crystalline form of any one of Embodiments A1 to A47.

Embodiment T6 is a further embodiment of Embodiment T1 wherein at least 50% of the tris salt of Compound 1 is present as the crystalline form of any one of Embodiments A1 to A47.

Embodiment T7 is a further embodiment of Embodiment T1 wherein at least 60% of the tris salt of Compound 1 is present as the crystalline form of any one of Embodiments A1 to A47.

Embodiment T8 is a further embodiment of Embodiment T1 wherein at least 70% of the tris salt of Compound 1 is present as the crystalline form of any one of Embodiments A1 to A47.

Embodiment T9 is a further embodiment of Embodiment T1 wherein at least 80% of the tris salt of Compound 1 is present as the crystalline form of any one of Embodiments A1 to A47.

Embodiment T10 is a further embodiment of Embodiment T1 wherein at least 90% of the tris salt of Compound 1 is present as the crystalline form of any one of Embodiments A1 to A47.

Embodiment T11 is a further embodiment of Embodiment T1 wherein at least 95% of the tris salt of Compound 1 is present as the crystalline form of any one of Embodiments A1 to A47.

Embodiment T12 is a further embodiment of Embodiment T1 wherein at least 97% of the tris salt of Compound 1 is present as the crystalline form of any one of Embodiments A1 to A47.

Embodiment T13 is a further embodiment of Embodiment T1 wherein at least 99% of the tris salt of Compound 1 is present as the crystalline form of any one of Embodiments A1 to A47.

Embodiment T14 is a further embodiment of any one of Embodiments T1 to T13, wherein the additional agent is(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof.

Embodiment T15 is a further embodiment of any one of Embodiments T1 to T13, wherein the additional agent is(S)-2-(5-((3-Ethoxypyridin-2-yl)oxy)pyridin-3-yl)-N-(tetrahydrofuran-3-yl)pyrimidine-5-carboxamide.

Embodiment T16 is a further embodiment of any one of Embodiments T1 to T13, wherein the additional agent is a crystalline solid form of(S)-2-(5-((3-Ethoxypyridin-2-yl)oxy)pyridin-3-yl)-N-(tetrahydrofuran-3-yl)pyrimidine-5-carboxamide.

Embodiment T17 is a further embodiment of any one of Embodiments T1 to T13, wherein the additional agent is Form 1 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment T18 is a further embodiment of any one of Embodiments T1 to T13, wherein the additional agent is Form 2 of(S)-2-(5-((3-Ethoxypyridin-2-yl)oxy)pyridin-3-yl)-N-(tetrahydrofuran-3-yl)pyrimidine-5-carboxamide (as described in Example 1 of U.S. Pat. No. 10,071,992).

Embodiment T19 is a further embodiment of any one of Embodiments T1 to T13, wherein the additional agent is 4-(4-(1-Isopropyl-7-oxo-1,4,6,7-tetrahydrospiro [indazole-5,4′-piperidine]-1′-carbonyl)-6-methoxypyridin-2-yl)benzoic acid, or a pharmaceutically acceptable salt thereof.

Embodiment T20 is a further embodiment of any one of Embodiments T1 to T13, wherein the additional agent is tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment T21 is a further embodiment of any one of Embodiments T1 to T13, wherein the additional agent is a crystalline tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid.

Embodiment T22 is a further embodiment of any one of Embodiments T1 to T13, wherein the additional agent is an anhydrous crystalline form (Form 1) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment T23 is a further embodiment of any one of Embodiments T1 to T13, wherein the additional agent is a trihydrate crystalline form (Form 2) of tris salt of 4-(4-(1-Isopropyl-7-oxo-1,4,6,7-tetrahydrospiro [indazole-5,4′-piperidine]-1′-carbonyl)-6-methoxypyridin-2-yl)benzoic acid (as described in International PCT Application No. PCT/IB2018/058966, published as WO2019102311).

Embodiment T24 is a further embodiment of any one of Embodiments T1 to T23, wherein the pharmaceutical combination comprising the tris salt of Compound 1 and the additional agent is a fixed-dose combination.

Embodiment T25 is a further embodiment of any one of Embodiments T1 to T23, wherein the pharmaceutical combination comprising the tris salt of Compound 1 and the additional agent is not a fixed-dose combination.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or lgs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Liposomes containing these agents and/or compounds of the invention are prepared by methods known in the art, such as described in U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

These agents and/or the compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing the compound of Formulas I, II, III, IV, or V, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a compound of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Kits

Another aspect of the invention provides kits comprising a solid form of the invention (e.g. Form 1 or Form 2) or pharmaceutical compositions comprising a solid form of the invention (e.g. Form 1 or Form 2). A kit may include, in addition to a solid form of the invention (e.g. Form 1 or Form 2) or pharmaceutical composition thereof, diagnostic or therapeutic agents. A kit may also include instructions for use in a diagnostic or therapeutic method. In some embodiments, the kit includes a crystalline form of the invention and a diagnostic agent. In other embodiments, the kit includes a crystalline form of the invention, or a pharmaceutical composition thereof.

In yet another embodiment, the invention comprises kits that are suitable for use in performing the methods of treatment described herein. In one embodiment, the kit contains a first dosage form comprising one or more of solid forms of the invention (e.g. Form 1 or Form 2) in quantities sufficient to carry out the methods of the invention. In another embodiment, the kit comprises one or more solid forms of the invention (e.g. Form 1 or Form 2) in quantities sufficient to carry out the methods of the invention and a container for the dosage and a container for the dosage.

### Preparation

Compound 1, tris salt thereof, and solid forms of tris salt of Compound 1, may be prepared by the general and specific methods described below, coupled with the common general knowledge of one skilled in the art of synthetic organic chemistry and/or solid forms of pharmaceutical compounds. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds, salts, and solid forms (e.g. crystalline form and amorphous) of the invention, it is noted that some of the preparation methods described herein may require protection of remote functionality (e.g., primary amine, secondary amine, carboxyl in precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and can typically be removed without chemically altering other functionality in the compounds.

The descriptions below are intended to provide a general description of the methodology employed in the preparation of the compounds and solid forms of the present invention. Some of the compounds of the present invention may contain single or multiple chiral centers with the stereochemical designation (R) or(S). It will be apparent to one skilled in the art that all of the synthetic transformations can be conducted in a similar manner whether the materials are enantioenriched or racemic. Moreover the resolution to the desired optically active material may take place at any desired point in the sequence using well known methods such as described herein and in the chemistry literature. For example, intermediates and finals may be separated using chiral chromatographic methods. Alternatively, chiral salts may be utilized to isolate enantiomerically enriched intermediates and final compounds.

### Examples

The following illustrate the synthesis of non-limiting compounds (including solid forms thereof) of the present invention.

Experiments were generally carried out under inert atmosphere (nitrogen or argon), particularly in cases where oxygen- or moisture-sensitive reagents or intermediates were employed. Commercial solvents and reagents were generally used without further purification. Anhydrous solvents were employed where appropriate, generally AcroSeal® products from Acros Organics, Aldrich® Sure/Seal™ from Sigma-Aldrich, or DriSolv® products from EMD Chemicals. In other cases, commercial solvents were passed through columns packed with 4 Å molecular sieves, until the following QC standards for water were attained: a) <100 ppm for dichloromethane, toluene, N, N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. The symbol + denotes that the chlorine isotope pattern was observed in the mass spectrum.

Chiral separations were used to separate enantiomers or diastereomers of some intermediates during the preparation of the compounds of the invention.

Reactions proceeding through detectable intermediates were generally followed by LCMS, and allowed to proceed to full conversion prior to addition of subsequent reagents. For syntheses referencing procedures in other Examples or Methods, reaction conditions (reaction time and temperature) may vary. In general, reactions were followed by thin-layer chromatography or mass spectrometry, and subjected to work-up when appropriate. Purifications may vary between experiments: in general, solvents and the solvent ratios used for eluents/gradients were chosen to provide appropriate Res or retention times. All starting materials in these Preparations and Examples are either commercially available or can be prepared by methods known in the art or as described herein.

### Example 1. Preparation of Form 1 of 1,3-dihydroxy-2-(hydroxymethyl) propan-2-amine salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (Form 1 of Tris salt of Compound 1)

2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid can be prepared, for example, as described in U.S. Pat. No. 10,208,019 (see Example 4A-01 of the patent).

2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid (solid, 1500 mg, 2.70 mmol) was added to a round bottom flask with a stir bar. 1-Propanol (131.0 mL) was added, and the flask heated with stirring to 70° C. An aqueous solution of Tris(hydroxymethyl)aminomethane (2.00 M, 1.42 mL, 2.84 mmol) was added dropwise. The mixture remained homogenous during the addition. After the addition was complete stirring was continued for a few minutes, and then the heat was turned down to about 55° C. After three hours, the heat was turned off and the sample was allowed to cool to room temperature overnight without stirring in the heat block covered with aluminium foil. After sitting overnight, no solid had formed. The side wall of the flask was scratched with a spatula. After a few minutes of stirring at room temperature, a solid had formed. The solid was stirred at room temperature for 30 minutes and then placed in an 80° C. oil bath for 10 minutes. The mixture was allowed to cool to room temperature and stir for an additional 3 hours. The white crystalline solid was collected with vacuum filtration and dried in a vacuum oven at room temperature overnight (1600 mg, 2.364 mmol, yield: 87.6%).

### Example 2. Alternative Preparation of Form 1 of Tris Salt of Compound 1

To a reaction vessel was added DMSO (16 mL) and the solvent was held at 25° C. Then to the vessel was added water (2 mL) and the mixed solvent was held at 25° C. To the vessel was added Tris salt of Compound 1 (2.0 g, 2.96 mmol) maintaining a temperature of 25° C.±5.0° C. The resultant mixture was heated to 65° C. over 30 minutes and then was held at 65° C. for a minimum of 30 minutes. To the vessel was slowly added water (2 mL) over 30 minutes while maintaining the temperature at 65° C. The resultant solution/mixture was then held at 65° C. for a minimum of 30 minutes. To the vessel was added seed crystals of Form 1 of tris salt of Compound 1 (10 mg, 0.01 mmol, see e.g. Example 1) while maintaining the temperature at 65° C. The mixture then was held at 65° C. for a minimum of 1 hour, and then was cooled to 30° C. at a rate of about 0.2° C./min. The mixture was then held at 30° C. for 1 hour. Then the mixture was heated to 45° C. over 30 minutes. The mixture was held at 45° C. and then was cooled to 15° C. at a rate of about 0.2° C./min. The mixture was held at 15° C. for at least 8 hours and then was filtered, and the resultant cake was air-dried. The cake was then washed with butan-2-one (MEK or methyl ethyl ketone, 6 mL, pre-cooled to 15° C.) and then allowed to air dry. Then the resultant crystalline solid was further dried in a vacuum oven at 55° C. for at least 8 hours.

The dried crystalline solid (Form 1) was determined to contain reduced amount of one or more of the following impurities (a compound of Formula IMP-1, IMP-2, or IMP-3, or a salt thereof) comparing the starting materials for the recrystallization process (using the DMSO/H₂O solvent system).

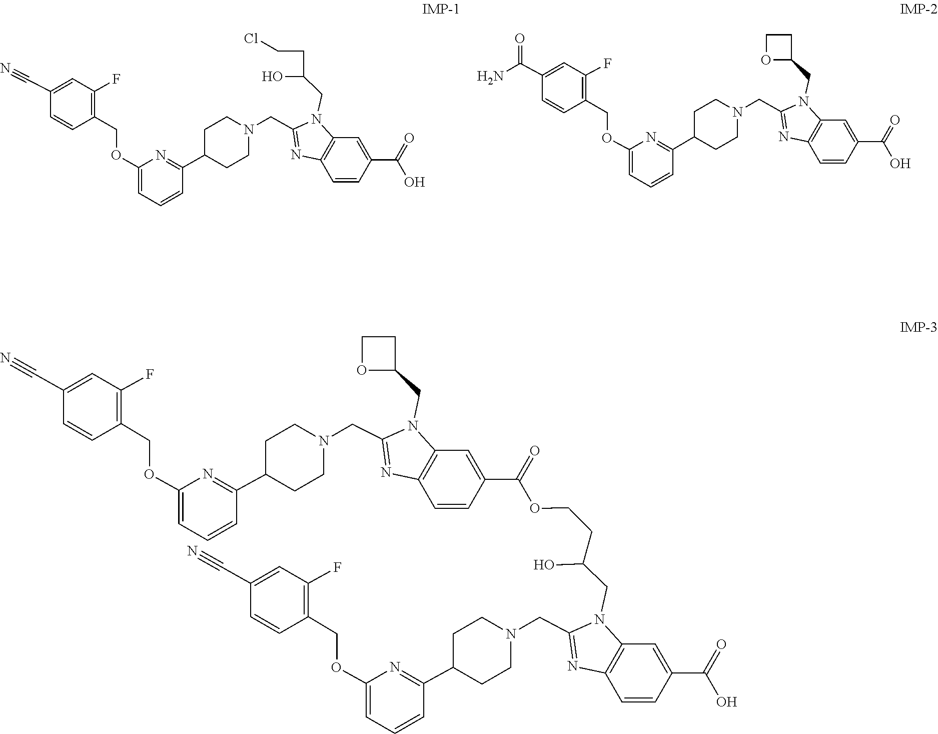 [SMILES: N#Cc1ccc(COc2cccc(C3CCN(Cc4nc5ccc(C(=O)O)cc5n4CC(O)CCCl)CC3)n2)c(F)c1 ; NC(=O)c1ccc(COc2cccc(C3CCN(Cc4nc5ccc(C(=O)O)cc5n4C[C@@H]4CCO4)CC3)n2)c(F)c1 ; N#Cc1ccc(COc2cccc(C3CCN(Cc4nc5ccc(C(=O)O)cc5n4CC(O)CCOC(=O)c4ccc5nc(CN6CCC(c7cccc(OCc8ccc(C#N)cc8F)n7)CC6)n(C[C@@H]6CCO6)c5c4)CC3)n2)c(F)c1]

In some embodiments, the crystalline Form 1 contains no more than about 1.0%, about 0.5%, about 0.4%, about 0.3%, about 0.2%, about 0.1%, or about 0.05% by weight of a compound of Formula IMP-1 or a salt thereof.

In some embodiments, the crystalline Form 1 contains no more than about 1.0%, about 0.5%, about 0.4%, about 0.3%, about 0.2%, about 0.1%, or about 0.05% by weight of a compound of Formula IMP-2 or a salt thereof.

In some embodiments, the crystalline Form 1 contains no more than about 1.0%, about 0.5%, about 0.4%, about 0.3%, about 0.2%, or about 0.1%, about 0.05%, or about 0.025% by weight of a compound of Formula IMP-3 or a salt thereof.

### Example 3. Powder X-Ray Diffraction Analysis of Form 1 of Tris Salt of Compound 1

The powder X-ray diffraction pattern for Form 1 was generated using a Bruker AXS D8 Endeavor diffractometer equipped with a copper (Cu) Kα radiation source, wavelength of 1.5406 Å. The tube voltage and amperage were set to 40 kV and 40 mA, respectively. The motorized divergence slits were set at constant illumination of 11 mm. Diffracted radiation was detected using a LYNXEYE XE-T energy dispersive X-ray detector, with the position sensitive detector (PSD) opening set at 4.00°. Data were collected on the theta-theta goniometer at the Cu wavelength from 2.0 to 55.0 degrees 2-theta (2θ) using a step size of 0.019 2θ and a time per step of 0.2 s. Samples were prepared for analysis by placing them in a silicon low background small divot holder and rotated at 15 rpm during data collection. Data were collected using Bruker DIFFRAC Plus software and analysis was performed by DIFFRAC.EVA V5.0 software.

Peak lists were prepared using reflections with a relative intensity ≥5% of the most intense band in each respective diffraction pattern. A typical error of ±0.2 2θ in peak positions (USP-941) applies to this data. The minor error associated with this measurement can occur because of a variety of factors including: (a) sample preparation (e.g. sample height), (b) instrument characteristics, (c) instrument calibration, (d) operator input (e.g. in determining the peak locations), and (e) the nature of the material (e.g. preferred orientation and transparency effects).

To obtain the absolute peak positions, the powder pattern should be aligned against a reference. This could either be the simulated powder pattern from the crystal structure of the same form solved at room temperature, or an internal standard e.g. silica or corundum. The collected powder pattern of Form 1 of tris salt of Compound 1 was aligned to the simulated powder pattern from the crystal structure. The PXRD pattern for Form 1 of tris salt of Compound 1 is provided in FIG. 1 and the corresponding peak list is provided in Table 1.

For PXRD, the relative intensities of the peaks can vary, depending upon, for example, the sample preparation technique and the sample mounting procedure. Moreover, instrument variation and other factors can often affect the 2-theta values. Therefore, the peak position assignments of diffraction patterns for Form 1, which may be used to characterize a crystalline material, can vary by plus or minus about 0.2°.

TABLE 1
PXRD peak list for Form 1
of tris salt of Compound 1

| Angle | Rel. |
|---|---|
| (°2θ) | Intensity (%) |
| 7.1 | 31 |
| 8.9 | 18 |
| 10.3 | 13 |
| 10.4 | 12 |
| 12.3 | 8 |
| 13.7 | 28 |
| 14.3 | 100 |
| 17.1 | 10 |
| 17.5 | 65 |
| 18.0 | 31 |
| 18.4 | 14 |
| 19.4 | 15 |
| 19.8 | 22 |
| 20.2 | 22 |
| 20.6 | 41 |
| 21.0 | 70 |
| 21.5 | 11 |
| 21.9 | 7 |
| 22.1 | 12 |
| 23.4 | 35 |
| 23.6 | 12 |
| 24.0 | 15 |
| 24.4 | 12 |
| 24.7 | 27 |
| 25.0 | 7 |
| 25.1 | 7 |
| 25.4 | 8 |
| 25.6 | 5 |
| 26.0 | 15 |
| 26.5 | 6 |
| 27.0 | 5 |
| 27.5 | 56 |
| 28.6 | 19 |
| 28.8 | 16 |
| 29.6 | 20 |
| 30.0 | 5 |

### Example 4. Solid State NMR Analysis of Form 1 of Tris Salt of Compound 1

Solid state NMR (ssNMR) analysis was conducted on a Bruker Avance III HD 400 MHZ (¹H frequency) NMR spectrometer. A 4 mm magic angle spinning (MAS) probe at MAS rates of 10 KHz was used for the ¹³C analysis. A ¹⁹F spectrum was recorded using a 3.2 mm MAS probe with a spin rate of 20 KHz. ¹⁵N ssNMR analysis was conducted on a Bruker AVANCE NEO 400 MHz NMR spectrometer equipped with a 4 mM MAS probe with a spin rate of 20 KHz. All spectra were acquired with the temperature was regulated to 20° C.

A ¹³C cross-polarization (CP) measurement with TOSS spinning sideband suppression was recorded with a 4 ms CP contact time and recycle delay of 40 s (see FIG. 2 and Table 2). A phase modulated proton decoupling field of ˜100 kHz was applied during spectral acquisition. Carbon spectral referencing is relative to neat tetramethylsilane, carried out by setting the high-frequency signal from an external sample of adamantane to 38.5 ppm.

A ¹⁹F spectrum was collected by direct excitation with proton decoupling and a 120 s recycle delay (see FIG. 3 and Table 3). Spectral referencing is with respect to CFCl₃, carried out by setting the resonance from an external sample of 50% v/v trifluoroacetic acid in H₂O to −76.54 ppm.

A ¹⁵N CP spectrum was recorded with a 10 ms CP contact time and a recycle delay of 3 s (see FIG. 4 and Table 4). Nitrogen spectral referencing is relative to neat nitromethane, carried out by setting the signal from an external sample of glycine to −346.8 ppm.

TABLE 2
¹³C ssNMR peak list for Form
1 of Tris salt of Compound 1.

| ¹³C Chemical | Relative |
|---|---|
| Shifts δ (ppm) | Intensity (%) |
| 24.7 | 16.6 |
| 26.9 | 17.6 |
| 31.5 | 37.4 |
| 43.9 | 37.3 |
| 50.9 | 29.3 |
| 53.0 | 21.7 |
| 54.6 | 34.2 |
| 55.8 | 19.5 |
| 57.0 | 44.4 |
| 59.2 | 71.2 |
| 64.0 | 57.0 |
| 67.8 | 21.9 |
| 68.2 | 21.6 |
| 81.1 | 26.1 |
| 82.7 | 28.4 |
| 109.4 | 67.2 |
| 112.6 | 28.0 |
| 113.7 | 16.7 |
| 115.4 | 18.2 |
| 116.6 | 23.1 |
| 117.7 | 68.1 |
| 119.4 | 29.3 |
| 120.7 | 26.0 |
| 123.9 | 51.9 |
| 130.0 | 100.0 |
| 133.1 | 58.4 |
| 135.2 | 28.6 |
| 137.4 | 33.7 |
| 139.0 | 31.3 |
| 141.3 | 34.8 |
| 141.9 | 34.9 |
| 153.6 | 53.4 |
| 156.3 | 39.9 |
| 158.8 | 27.4 |
| 160.4 | 31.8 |
| 161.5 | 79.7 |
| 171.0 | 63.7 |

TABLE 3
¹⁵N ssNMR peak list for Form
1 of Tris salt of Compound 1.

| ¹⁵N Chemical | Relative |
|---|---|
| Shifts δ (ppm) | Intensity (%) |
| −339.9 | 100.0 |
| −338.2 | 70.3 |
| −335.1 | 65.9 |
| −223.4 | 53.4 |
| −147.7 | 25.1 |
| −144.7 | 21.0 |
| −120.9 | 31.4 |
| −117.2 | 36.5 |

TABLE 4
¹⁹F ssNMR peak list for Form 1
of Tris salt of Compound 1.

| ¹⁹F Chemical | Relative |
|---|---|
| Shifts δ (ppm) | Intensity (%) |
| −118.8 | 100.0 |
| −118.1 | 96.6 |

Peak positions and relative intensities were obtained using ACD Labs Spectrus Processor 2019 software. The error in the reported peak positions in the ¹³C, ¹⁵N and ¹⁹F ssNMR data is estimated to be ±0.2 ppm. The ssNMR intensities can vary depending on the setup of the experimental parameters and the thermal history of the sample.

### Example 5. Raman Spectroscopy Analysis of Form 1 of Tris Salt of Compound 1

Raman spectra were collected using a RAM II FT-Raman module attached to a Vertex 70 spectrometer (Bruker Optik GmbH). The instrument was equipped with a 1064 nm solid-state (Nd:YAG) laser and a liquid nitrogen cooled germanium detector. Prior to data acquisition, instrument performance and calibration verifications were conducted using a white light source, and polystyrene and naphthalene references.

Samples were prepared and analyzed in truncated NMR tubes. A sample rotator (Ventacon, UK) was used during measurement to maximise the volume of material exposed to the laser during data collection. The backscattered Raman signal from the sample was optimised and data were collected at a spectral resolution of 2 cm⁻¹ using a laser power of 500 mW. A Blackmann-Harris 4-term apodization function was applied to minimise spectral aberrations. Spectra were generated between 3500 and 50 cm⁻¹ with the number of scans adjusted accordingly to ensure adequate signal to noise.

Spectra were normalized by setting the intensity of the most intense peak to 2.00. Peaks were then identified using the automatic peak picking function in the OPUS v8.2 software (Bruker Optik GmbH) with the sensitivity set to 2%. Peak positions and relative peak intensities were extracted and tabulated. The variability in the peak positions with this experimental configuration is within +2 cm⁻¹.

FIG. 5 shows a representative FT-Raman spectrum of Form 1 of Tris salt of Compound 1 collected and Table 5 shows the FT-Raman peak list for Form 1 of Tris salt of Compound 1

TABLE 5
FT-Raman peak list for Form 1
of Tris salt of Compound 1.

| Wavenumber (cm⁻¹) | Relative |
|---|---|
| ±2 cm⁻¹ | Intensity (%) |
| 212 | 15.0 |
| 240 | 13.1 |
| 296 | 8.8 |
| 416 | 14.5 |
| 430 | 9.6 |
| 479 | 4.9 |
| 539 | 6.2 |
| 564 | 4.0 |
| 624 | 5.1 |
| 676 | 6.6 |
| 702 | 7.8 |
| 763 | 9.8 |
| 782 | 13.3 |
| 798 | 11.8 |
| 813 | 12.1 |
| 888 | 6.5 |
| 939 | 6.3 |
| 990 | 22.7 |
| 1060 | 7.1 |
| 1082 | 10.3 |
| 1096 | 8.1 |
| 1119 | 7.6 |
| 1146 | 11.7 |
| 1160 | 11.7 |
| 1197 | 5.5 |
| 1217 | 9.5 |
| 1261 | 29.6 |
| 1284 | 18.4 |
| 1334 | 11.6 |
| 1347 | 11.5 |
| 1371 | 29.1 |
| 1417 | 6.5 |
| 1459 | 17.5 |
| 1509 | 31.5 |
| 1576 | 11.4 |
| 1624 | 54.1 |
| 2205 | 5.2 |
| 2232 | 100.0 |
| 2761 | 9.3 |
| 2803 | 13.0 |
| 2878 | 13.1 |
| 2905 | 24.0 |
| 2937 | 24.3 |
| 2952 | 19.1 |
| 3026 | 8.7 |
| 3055 | 7.5 |
| 3082 | 13.8 |

### Example 6. Preparation of Form 2 of Tris Salt of Compound 1

Form 1 of tris salt of Compound 1 (Example 2, 2 g), was added into a 35 mL ball mill jar. A 20 mm ball bearing was placed into the ball mill jar. The milling jar was sealed and placed on a Retsch MM400 ball mill where the sample was milled for 60 minutes at 30 Hz and ambient temperature. The resultant solid is designated as Form 2 of tris salt of Compound 1.

### Example 7. Powder X-Ray Diffraction Analysis of Form 2 of Tris Salt of Compound 1

The powder X-ray diffraction pattern for Form 2 (an amorphous form) was generated using a Bruker AXS D8 Endeavor diffractometer equipped with a copper (Cu) Kα radiation source, wavelength of 1.5406 Å. The tube voltage and amperage were set to 40 kV and 40 mA, respectively. The motorized divergence slits were set at constant illumination of 11 mm. Diffracted radiation was detected using a LYNXEYE XE-T energy dispersive X-ray detector, with the position sensitive detector (PSD) opening set at 4.00. Data was collected on the theta-theta goniometer at the Cu wavelength from 2.0 to 55.0 degrees 2-theta (20) using a step size of 0.019 2θ and a time per step of 0.2 s. Samples were prepared for analysis by placing them in a silicon low background small divot holder and rotated at 15 rpm during data collection. Data were collected using Bruker DIFFRAC Plus software and analysis was performed by DIFFRAC.EVA V5.0 software.

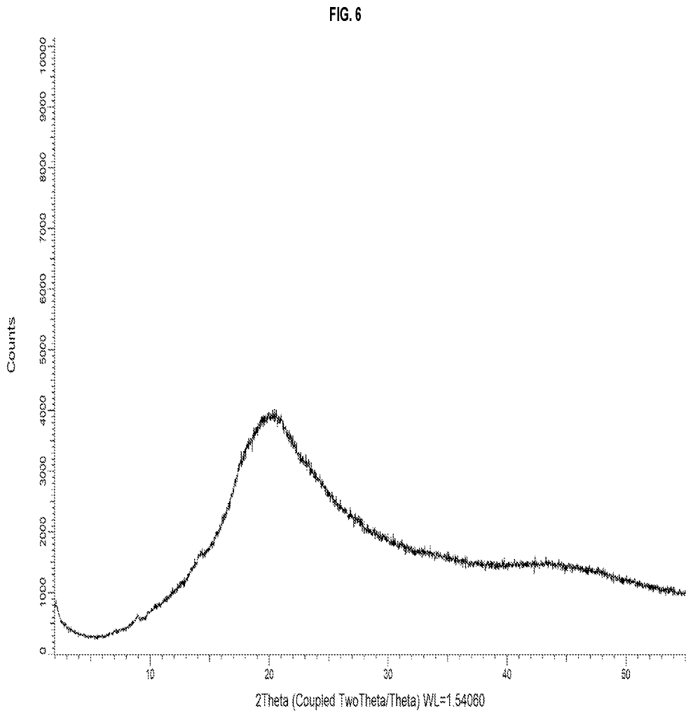

The PXRD profile collected for Form 2 is provided in FIG. 6, which is typical for amorphous material, i.e., it does not give distinctive powder X-ray diffraction patterns (not having any sharp peaks as in a PXRD for Form 1).

In some embodiments, Form 2 has a PXRD substantially the same as FIG. 6.

### Example 8. Solid State NMR Analysis of Form 2 of Tris Salt of Compound 1

Solid state NMR (ssNMR) analysis was conducted on a Bruker Avance III HD 400 MHZ (¹H frequency) NMR spectrometer. A 4 mm magic angle spinning (MAS) probe at MAS rates of 10 KHz and 8 KHz was used for ¹³C and ¹⁵N spectra, respectively. A ¹⁹F spectrum was recorded using the same spectrometer and a 3.2 mm MAS probe with a spin rate of 20 KHz. All spectra were acquired with the temperature was regulated to 20° C.

A ¹³C cross-polarization (CP) measurement with TOSS spinning sideband suppression was recorded with a 1 ms CP contact time and recycle delay of 2 s (See FIG. 7). A phase modulated proton decoupling field of ˜100 kHz was applied during spectral acquisition. Carbon spectral referencing is relative to neat tetramethylsilane, carried out by setting the high-frequency signal from an external sample of adamantane to 38.5 ppm. A ¹⁵N CP spectrum was recorded with a 4 ms CP contact time and a recycle delay of 2 s (See FIG. 8). Nitrogen spectral referencing is relative to neat nitromethane, carried out by setting the signal from an external sample of glycine to −346.8 ppm. A ¹⁹F spectrum was collected by direct excitation with proton decoupling and a 10 s recycle delay (See FIG. 9). Spectral referencing is with respect to CFCl₃, carried out by setting the resonance from an external sample of 50% v/v trifluoroacetic acid in H₂O to −76.54 ppm.

Peak positions and relative intensities were obtained using ACD Labs Spectrus Processor 2019 software to provide the relative intensities for each peak. Due to the relatively high line width for a number of peaks, combined with resonance overlaps and noise levels there is an estimated error of between ±0.2 and 0.5 ppm range for peak positions in the ¹³C spectrum (see Table 6). Estimated errors of between ±0.8 and 1.5 ppm are provided for peak positions in the ¹⁵N and ¹⁹F spectra (See Tables 7 and 8).

TABLE 6
¹³C ssNMR peak list for Form
2 of tris salt of Compound 1

| ¹³C chemical | |
|---|---|
| shift δ (ppm) | |
| Estimated error | |
| is ±0.2 ppm | Relative |
| unless stated | Intensity |
| otherwise | (%) |
| 25.0 ± 0.3 | 28.8 |
| 29.6 | 31.9 |
| 32.0 ± 0.3 | 29.0 |
| 33.9 ± 0.5 | 26.4 |
| 43.8 | 33.0 |
| 55.4 ± 0.3 | 63.3 |
| 60.1 ± 0.5 | 51.1 |
| 62.2 ± 0.3 | 56.0 |
| 68.3 | 36.2 |
| 81.6 ± 0.3 | 27.7 |
| 112.6 | 68.6 |
| 117.9 | 71.4 |
| 124.4 ± 0.5 | 32.3 |
| 130.8 ± 0.3 | 71.4 |
| 132.5 ± 0.3 | 69.8 |
| 135.6 | 59.0 |
| 143.9 ± 0.3 | 40.4 |
| 154.1 | 42.1 |
| 157.1 ± 0.5 | 28.7 |
| 162.2 | 100.0 |
| 174.2 | 26.0 |

TABLE 7
¹⁵N ssNMR peak list for Form
2 of tris salt of Compound 1

| ¹⁵N chemical | Relative |
|---|---|
| shifts δ (ppm) | Intensity (%) |
| −332.7 ± 0.8 | 100 |
| −229 ± 1.0 | 56.4 |
| −147 ± 1.5 | 17.3 |
| −118 ± 1.5 | 30.0 |

TABLE 8
¹⁹F ssNMR peak list for Form
2 of tris salt of Compound 1

| ¹⁹F chemical | Relative |
|---|---|
| shifts δ (ppm) | Intensity (%) |
| −116.3 ± 0.8 | 100.0 |

### Example 9. Raman Spectroscopy Analysis of Form 2 of Tris Salt of Compound 1

Raman spectra were collected using a RAM II FT-Raman module attached to a Vertex 70 spectrometer (Bruker Optik GmbH). The instrument is equipped with a 1064 nm solid-state (Nd:YAG) laser and a liquid nitrogen cooled germanium detector. Prior to data acquisition, instrument performance and calibration verifications were conducted using a white light source, and polystyrene and naphthalene references.

Samples were prepared and analysed in truncated NMR tubes. A sample rotator (Ventacon, UK) was used during measurement to maximize the volume of material exposed to the laser during data collection. The backscattered Raman signal from the sample was optimized and data were collected at a spectral resolution of 2 cm⁻¹ using a laser power of 500 mW. A Blackmann-Harris 4-term apodization function was applied to minimise spectral aberrations. Spectra were generated between 3500 and 50 cm 1 with the number of scans adjusted accordingly to ensure adequate signal to noise.

Spectra were normalized by setting the intensity of the most intense peak to 2.00. Peaks were then identified using the automatic peak picking function in the OPUS v8.2 software (Bruker Optik GmbH) with the sensitivity set to 2%. Peak positions and relative peak intensities were extracted and tabulated. The variability in the peak positions with this experimental configuration is within ±2 cm⁻¹.

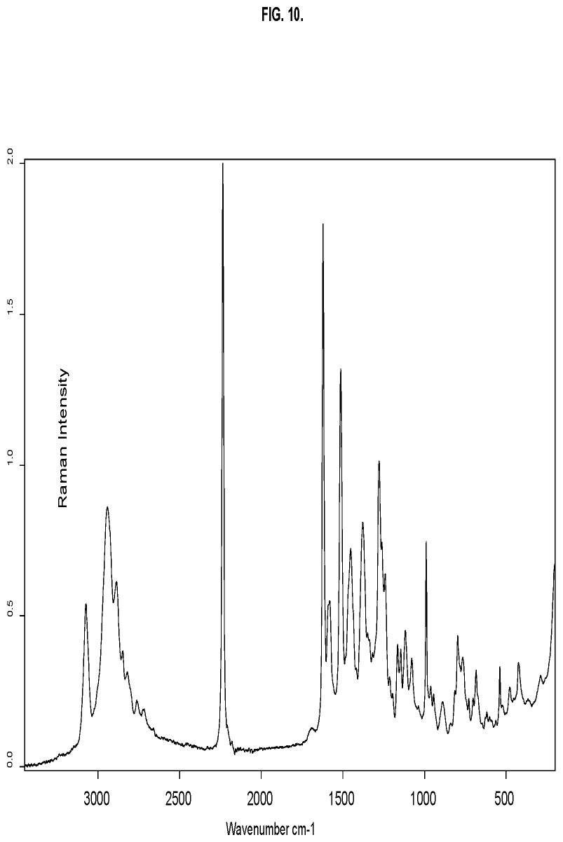

FIG. 10 shows a representative FT-Raman spectrum of Form 2 of Tris salt of Compound 1 collected and Table 9 shows the FT-Raman peak list for Form 2 of Tris salt of Compound 1

TABLE 9
Raman peak list for Form
2 of Tris salt of Compound 1.

| Wavenumber (cm⁻¹) | Relative |
|---|---|
| ±2 cm⁻¹ | Intensity (%) |
| 424 | 17.3 |
| 478 | 13.1 |
| 538 | 16.6 |
| 618 | 9.1 |
| 683 | 16.0 |
| 765 | 18.1 |
| 796 | 21.7 |
| 888 | 10.7 |
| 989 | 37.3 |
| 1078 | 18.0 |
| 1117 | 22.5 |
| 1145 | 19.4 |
| 1164 | 20.2 |
| 1240 | 31.9 |
| 1278 | 50.6 |
| 1378 | 40.5 |
| 1452 | 36.1 |
| 1513 | 66.0 |
| 1580 | 27.5 |
| 1621 | 90.3 |
| 2235 | 100.0 |
| 2763 | 11.0 |
| 2886 | 30.6 |
| 2942 | 43.0 |
| 3074 | 27.0 |

### Example AA. CHO GLP-1R Clone H6—Assay 1

GLP-1R-mediated agonist activity was determined with a cell-based functional assay utilizing an HTRF (Homogeneous Time-Resolved Fluorescence) CAMP detection kit (cAMP HI Range Assay Kit; CisBio cat #62AM6PEJ) that measures CAMP levels in the cell. The method is a competitive immunoassay between native cAMP produced by the cells and exogenous CAMP labeled with the dye d2. The tracer binding is visualized by a mAb anti-cAMP labeled with Cryptate. The specific signal (i.e. energy transfer) is inversely proportional to the concentration of CAMP in either standard or experimental sample.

The human GLP-1R coding sequence (NCBI Reference Sequence NP_002053.3, including naturally-occurring variant Gly168Ser) was subcloned into pcDNA3 (Invitrogen) and a cell line stably expressing the receptor was isolated (designated Clone H6). Saturation binding analyses (filtration assay procedure) using ¹²⁵I-GLP-1₇₋₃₆ (Perkin Elmer) showed that plasma membranes derived from this cell line express a high GLP-1R density (K_{d}: 0.4 nM, Bₘₐₓ: 1900 fmol/mg protein).

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The cell pellet was then re-suspended in 10 mL of growth medium [DMEM/F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081) and 500 μg/mL Geneticin (G418) (Invitrogen #10131035)]. A 1 mL sample of the cell suspension in growth media was counted on a Becton Dickinson ViCell to determine cell viability and cell count per mL. The remaining cell suspension was then adjusted with growth media to deliver 2000 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment in 5% carbon dioxide.

Varying concentrations of each compound to be tested (in DMSO) were diluted in assay buffer (HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E) containing 100 UM 3-isobutyl-1-methylxanthin (IBMX; Sigma cat #15879). The final DMSO concentration is 1%.

After 48 hours, the growth media was removed from the assay plate wells, and the cells were treated with 20 μL of the serially diluted compound in assay buffer for 30 minutes at 37° C. in a humidified environment in 5% carbon dioxide. Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a CAMP standard curve (as described in the manufacturer's assay protocol) and the percent effect was determined relative to a saturating concentration of the full agonist GLP-1₇₋₃₆ (1 μM) included on each plate. EC₅₀ determinations were made from agonist dose-response curves analyzed with a curve fitting program using a 4-parameter logistic dose response equation.

### Example BB. CHO GLP-1R Clone C6—Assay 2

GLP-1R-mediated agonist activity was determined with a cell-based functional assay utilizing an HTRF (Homogeneous Time-Resolved Fluorescence) cAMP detection kit (cAMP HI Range Assay Kit; Cis Bio cat #62AM6PEJ) that measures cAMP levels in the cell. The method is a competitive immunoassay between native cAMP produced by the cells and exogenous CAMP labeled with the dye d2. The tracer binding is visualized by a mAb anti-CAMP labeled with Cryptate. The specific signal (i.e. energy transfer) is inversely proportional to the concentration of CAMP in either a standard or an experimental sample.

The human GLP-1R coding sequence (NCBI Reference Sequence NP_002053.3, including naturally-occurring variant Leu260Phe) was subcloned into pcDNA5-FRT-TO and a clonal CHO cell line stably expressing a low receptor density was isolated using the Flp-In™ T-Rex™ System, as described by the manufacturer (ThermoFisher). Saturation binding analyses (filtration assay procedure) using 125|-GLP-1 (Perkin Elmer) showed that plasma membranes derived from this cell line (designated clone C6) express a low GLP-1R density (K_{d}: 0.3 nM, Bₘₐₓ: 240 fmol/mg protein), relative to the clone H6 cell line.

Cells were removed from cryopreservation, re-suspended in 40 ml of Dulbecco's Phosphate Buffered Saline (DPBS—Lonza Cat #17-512Q) and centrifuged at 800×g for 5 minutes at 22° C. The DPBS was aspirated, and the cell pellet was re-suspended in 10 ml of complete growth medium (DMEM:F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081), 700 μg/mL Hygromycin (Invitrogen Cat #10687010) and 15 μg/mL Blasticidin (Gibco Cat #R21001). A 1 mL sample of the cell suspension in growth media was counted on a Becton Dickinson ViCell to determine cell viability and cell count per mL. The remaining cell suspension was then adjusted with growth media to deliver 1600 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment (95% O₂, 5% CO₂)

Varying concentrations of each compound to be tested (in DMSO) were diluted in assay buffer [HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E)] containing 100 μM 3-isobutyl-1-methylxanthin (IBMX; Sigma cat #15879). The final DMSO concentration in the compound/assay buffer mixture is 1%.

After 48 hours, the growth media was removed from the assay plate wells, and the cells were treated with 20 μL of the serially diluted compound in assay buffer for 30 minutes at 37° C. in a humidified environment (95% O₂, 5% CO₂). Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a CAMP standard curve (as described in the manufacturer's assay protocol) and the percent effect was determined relative to a saturating concentration of the full agonist GLP-1 (1 μM) included on each plate. EC₅₀ determinations were made from agonist dose response curves analyzed with a curve fitting program using a 4-parameter logistic dose response equation.

In Table X-1, assay data are presented to two (2) significant figures as the geometric mean (EC₅₀S) and arithmetic mean (Emax) based on the number of replicates listed (Number).

TABLE X-1
Biological activity for Compound 1.

| | Assay 1 | Assay 1 | | Assay 2 | Assay 2 | |
|---|---|---|---|---|---|---|
| | EC₅₀ | Emax | Assay 1 | EC₅₀ | Emax | Assay 2 |
| Compound | (nM) | (%) | Number | (nM) | (%) | Number |
| Compound 1 | 1.1 | 79 | 5 | 13 | 100 | 18 |

All patents, patent applications and references referred to herein are hereby incorporated by reference in their entirety.

What is claimed is:

1. A crystalline form of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt, wherein the crystalline form is Form 1, and wherein the crystalline form has a purity of greater than 90%.

2. The crystalline form of claim 1, and wherein the crystalline form has a powder X-ray diffraction pattern (PXRD) comprising one peak, in terms of 2θ (Cu Kα radiation source, wavelength of 1.5406 Å), selected from those at 14.3±0.2°, 17.5±0.2°, and 18.0±0.2°.

3. The crystalline form of claim 2, wherein the crystalline form has a powder X-ray diffraction pattern (PXRD) comprising peaks, in terms of 2θ, at 14.3±0.2°, 17.5±0.2°, and 18.0±0.2°.

4. The crystalline form of claim 2, wherein the crystalline form has a powder X-ray diffraction pattern (PXRD) comprising peaks, in terms of 2θ, at 14.3±0.2°, 17.5±0.2°, 18.0±0.2°, 23.4±0.2°, and 24.7 0.2°.

5. The crystalline form of claim 1 wherein the crystalline form has a ¹³C ssNMR spectrum comprising peaks, in terms of chemical shifts, at 171.0±0.2 ppm and 141.3±0.2 ppm.

6. The crystalline form of claim 1 wherein the crystalline form has a ¹⁵N ssNMR spectrum comprising peaks, in terms of chemical shifts, at −339.9±0.2 ppm and −223.4±0.2 ppm.

7. The crystalline form of claim 1 wherein the crystalline form has a ¹⁹F ssNMR spectrum comprising one peak, in terms of chemical shifts, at −118.8±0.2 ppm.

8. The crystalline form of claim 1 wherein the crystalline form has an FT-Raman spectrum comprising one peak, in terms of wavenumbers (cm⁻¹), selected from those at 1371±2 cm⁻¹, 430±2 cm⁻¹, and 416±2 cm⁻¹.

9. The crystalline form of claim 1 wherein the crystalline form has an FT-Raman spectrum comprising peaks, in terms of wavenumbers (cm⁻¹), at 1371±2 cm⁻¹, 430±2 cm⁻¹, and 416±2 cm⁻¹.

10. A pharmaceutical composition comprising a therapeutically effective amount of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and a pharmaceutically acceptable carrier, wherein at least 90% of the tris salt of Compound 1 is present as the crystalline form of claim 1.

11. An amorphous form of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt, wherein the amorphous is Form 2, and wherein the amorphous form has purity greater than 90%.

12. The amorphous form of claim 11, wherein the amorphous form has a ¹³C ssNMR spectrum comprising one peak, in terms of chemical shifts, selected from those at 174.0±0.2 ppm, 143.9±0.3 ppm, and 62.2±0.3 ppm.

13. The amorphous form of claim 11, wherein the amorphous form has a ¹³C ssNMR spectrum comprising peaks, in terms of chemical shifts, at 174.0±0.2 ppm, 143.9±0.3 ppm, and 62.2±0.3 ppm.

14. The amorphous form of claim 11 wherein the amorphous form has a ¹⁵N ssNMR spectrum comprising peaks, in terms of chemical shifts, at −332.7±0.8 ppm and −229±1.0 ppm.

15. The amorphous form of claim 11 wherein the amorphous form has a ¹⁹F ssNMR spectrum comprising one peak, in terms of chemical shifts, at −116.3±0.8 ppm.

16. The amorphous form of claim 11 wherein the amorphous form has an FT-Raman spectrum comprising one peak, in terms of wavenumbers (cm⁻¹), selected from those at 1513±2 cm⁻¹, 1278±2 cm⁻¹, and 1378±2 cm⁻¹.

17. A pharmaceutical composition comprising a therapeutically effective amount of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt (“tris salt of Compound 1”) and a pharmaceutically acceptable carrier, wherein at least 50% of the tris salt of Compound 1 is present as the amorphous form of claim 11.

18. A method for treating a disease or disorder in a human comprising administering the human the crystalline form of tris salt of Compound 1 of claim 1, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

19. A method for treating a disease or disorder in a human comprising administering the human the amorphous form of tris salt of Compound 1 of claim 11, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.
